(19) United States
(12) Patent Application Publication
(10) Pub. No.: US 2004/0006137 A1
(43) Pub. Date: Jan. 8, 2004

(54) Asymmetric synthesis of amino-pyrrolidinones and a crystalline, free-base amino-pyrrolidinone

(76) Inventors: Robert E. Waltermire, Hillsborough, NJ (US); Silvio Campagna, Candia, NH (US); Scott A. Savage, Yardley, PA (US); Shailendra Bordawekar, Newark, DE (US); Thomas P. Maduskuie, Wilmington, DE (US); Sridhar Desikan, Hillsborough, NJ (US); Stephen R. Anderson, Stonington, CT (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/389,597

(22) Filed: Mar. 14, 2003

Related U.S. Application Data
(60) Provisional application No. 60/392,440, filed on Jun. 28, 2002.
(60) Provisional application No. 60/400,411, filed on Aug. 1, 2002.

(51) Int. Cl. A61K 31/24; C07C 229/42

(52) U.S. Cl. 514/534; 560/39

(57) ABSTRACT

A novel process for the asymmetric synthesis of an amino-pyrrolidinone of the type shown below is described.

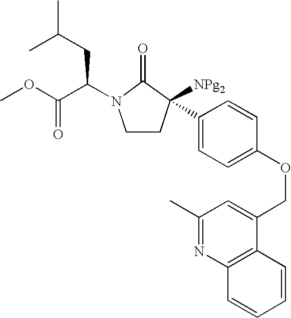

These compounds are useful as intermediates for MMP and TACE inhibitors.

Crystalline, free-base form of Compound J ((2R)-2-((3R)-3-amino-3-{-[(2-methyl-4-quinolinyl)methoxy]phenyl}-2-oxopyrrolidinyl)-N-hydroxy-4-methylpentanamide):

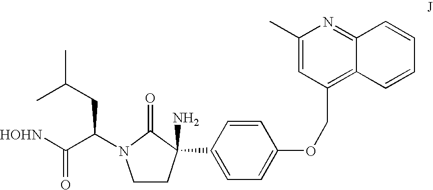

which is useful as a TACE inhibitor, pharmaceutical compositions comprising the same, and methods of using the same for treating inflammatory diseases are also described.

### CROSS-REFERENCE TO RELATED APPLICATIONS

This application claims the priority benefits of U.S. Provisional Application No. 60/392,440, filed Jun. 28, 2002, and U.S. Provisional Application No. 60/400,411, filed Aug. 1, 2002, all of which are fully incorporated herein by reference.

### FIELD OF THE INVENTION

The present invention relates generally to processes for the asymmetric synthesis of amino-pyrrolidinones, such pyrrolidinones being useful as intermediates for MMP and TACE inhibitors. This invention also relates generally to a novel crystalline, free-base form of Compound J, described below. Specifically, the potent TACE inhibitor, Compound J, can be produced as a crystalline free-base that exists in one form. The present invention also relates to pharmaceutical compositions comprising the same and methods of using the same.

### BACKGROUND OF THE INVENTION

Amino-pyrrolidinones (see Compound J below) are currently being studied as MMP and TACE inhibitors in clinical settings. Clinical trials and NDA submissions require practical, large-scale synthesis of the active drug.

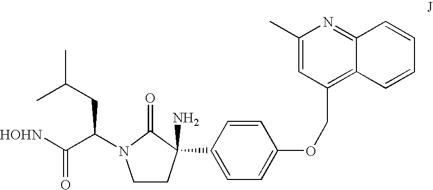

Consequently, it is desirable to find new synthetic procedures for making amino-pyrrolidinones.

The bis-trifluoroacetic acid salt of Compound J is disclosed as Example 356 in U.S. Pat. No. 6,057,336, the contents of which are hereby incorporated by reference. Compound J can be named in at least two ways: (a) (2R)-2-((3R)-3-amino-3-{[(2-methyl-4-quinolinyl)methoxy]phenyl}-2-oxopyrrolidinyl)-N-hydroxy-4-methylpentanamide or (b) [1-(R)]-3-amino-N-hydroxy-alpha-(2-methylpropyl)-3-[4-[(2-methyl-4-quinolinyl)methoxy]phenyl]-2-oxo-1-pyrrolidineacetamide).

Compound J has not been known previously to exist in a stable, neutral, crystalline form, only its bis-trifluoroacetic salt form. For the manufacture, purification, and formulation of drug substances, it is advantageous to discover a stable (e.g., non-hygroscopic) crystalline form of Compound J. Thus, the present invention also relates to a crystalline, free-base form of Compound J.

### SUMMARY OF THE INVENTION

Accordingly, the present invention provides a novel intermediate for making an amino-pyrrolidinone.

The present invention provides a novel amino-pyrrolidinone.

The present invention provides a novel process for making amino-pyrrolidinones.

These and other objects, which will become apparent during the following detailed description, have been achieved by the inventors' discovery that compounds of formula II can be formed from compounds of formula I.

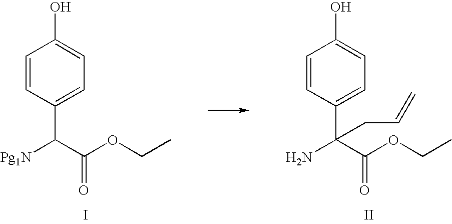

The present invention also provides a novel crystalline, free-base form of Compound J ((2R)-2-((3R)-3-amino-3-{-[(2-methyl-4-quinolinyl)methoxy]phenyl}-2-oxopyrrolidinyl)-N-hydroxy-4-methylpentanamide).

The present invention provides pharmaceutical compositions comprising a pharmaceutically acceptable carrier and a therapeutically effective amount of the crystalline form of Compound J.

The present invention provides a method for treating inflammatory disorders, comprising: administering to a host, in need of such treatment, a therapeutically effective amount of the crystalline form of Compound J.

The present invention provides a method of treating a condition or disease mediated by MMPs, TACE, or a combination thereof in a mammal, comprising: administering to the mammal in need of such treatment a therapeutically effective amount of the crystalline form of Compound J.

The present invention provides a method comprising: administering the crystalline form of Compound J in an amount effective to treat a condition or disease mediated by MMPs, TACE, or a combination thereof.

The present invention provides a method for treating inflammatory disorders, comprising: administering, in combination, to a host in need thereof, a therapeutically effective amount of:

(a) the crystalline form of Compound J; and,

(b) one or more additional anti-inflammatory agents selected from selective COX-2 inhibitors, interleukin-1 antagonists, dihydroorotate synthase inhibitors, p38 MAP kinase inhibitors, TNF-α inhibitors, TNF-α sequestration agents, and methotrexate.

The present invention provides novel compounds of the present invention for use in therapy.

The present invention provides the use of novel compounds of the present invention for the manufacture of a medicament for the treatment of a condition or disease mediated by MMPs, TACE, or a combination thereof.

These and other objects, which will become apparent during the following detailed description, have been achieved by the inventors' discovery that the novel crystalline, free-base form of Compound J:

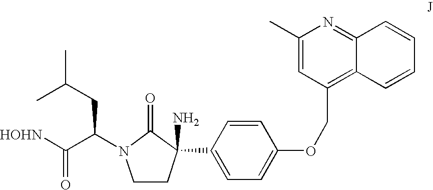

is an effective MMP and/or TACE inhibitor.

### BRIEF DESCRIPTION OF THE DRAWINGS

The invention is illustrated by reference to the accompanying drawings described below.

FIG. 1 shows a powder x-ray diffractogram of the anhydrous form of Compound J.

FIG. 2 shows a differential scanning calorimetry thermogram of the crystalline form of Compound J.

FIG. 3 shows a thermogravimetric analysis of Form I of Compound J.

### DETAILED DESCRIPTION OF THE INVENTION

[1] Thus, in an embodiment, the present invention provides a novel process, comprising:

(a) contacting a compound of formula I with a base and a halo-allyl group to form a compound of formula II:

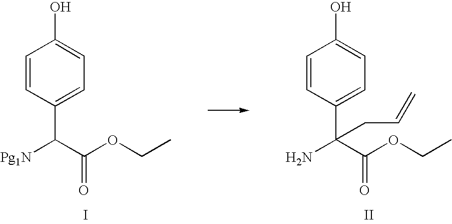

wherein: Pg₁ is an amino protecting group or represents H and an amino protecting group; and

the ratio of carbon to oxygen alkylation is from about 50-1000.

[2] In a preferred embodiment, the present invention provides a novel process further comprising: (b) resolving a compound of formula II to form a compound of formula III:

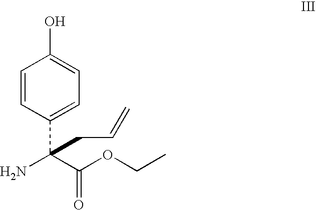

wherein: the base for (a) is lithium t-butoxide;

the allyl-halo reagent is allyl bromide; and

Pg₁ is 4-methylbenzaldehyde.

[3] In another preferred embodiment, the present invention provides a novel process wherein (b) is performed by contacting a compound of formula II with pig liver esterase and the enantiomeric excess of the R-isomer of formula III is at least 90%.

[4] In another preferred embodiment, the present invention provides a novel process further comprising:

(c) protecting the amino group of the compound of formula III to form a compound of formula IV:

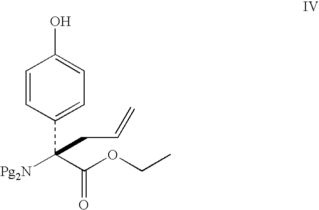

wherein Pg₂ is an amino protecting group or represents H and an amino protecting group.

[5] In another preferred embodiment, the present invention provides a novel process wherein (c) is performed by contacting a compound of formula III with an amino protecting reagent in the presence of a base;

wherein the amino protecting group reagent is selected form di-tert-butyl dicarbonate and 2-(tert-butoxycarbonyloxyimino)-2-phenylacetonitrile; and,

the base is selected from lithium carbonate, sodium carbonate, and potassium carbonate.

[6] In another preferred embodiment, the present invention provides a novel process wherein:

Pg₂ is tert-butoxycarbonyl;

the amino protecting group reagent is di-tert-butyl dicarbonate; and,

the base is lithium carbonate.

[7] In another preferred embodiment, the present invention provides a novel process further comprising:

(d) contacting a compound of formula IV with an alkylating agent to form a compound of formula V:

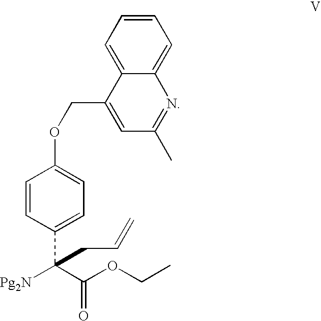

[8] In another preferred embodiment, the present invention provides a novel process wherein the alkylating agent is a 4-halomethyl-2-methylquinoline and the contacting is done in the presence of a base.

[9] In another preferred embodiment, the present invention provides a novel process wherein the alkylating agent is 4-chloromethyl-2-methylquinoline and the base is potassium tert-butoxide.

[10] In another preferred embodiment, the present invention provides a novel process further comprising:

(e) cleaving the olefin of formula V to form a compound of formula VI; and,

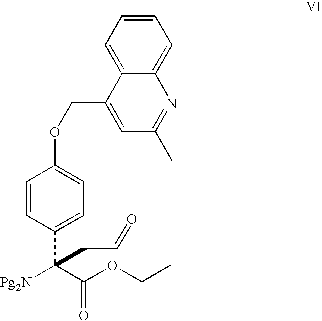

(f) contacting the compound of formula VI with an ester of an amino acid to form a compound of formula VII;

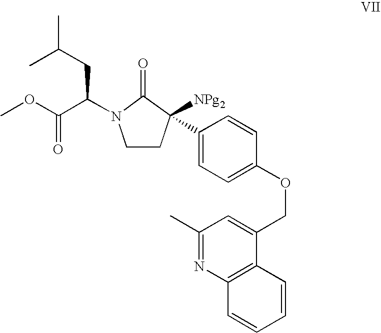

wherein (f) is done in the presence of a reducing agent.

[11] In another preferred embodiment, the present invention provides a novel process further comprising:

(g) deprotecting the compound of formula VII to form a compound of formula VIII.

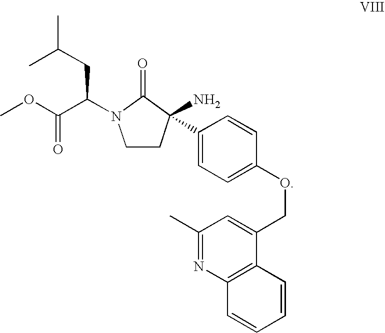

[12] In another embodiment, the present invention provides a novel compound of formula III:

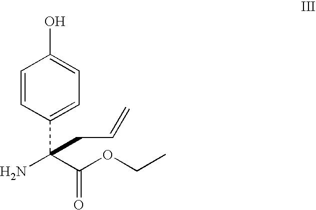

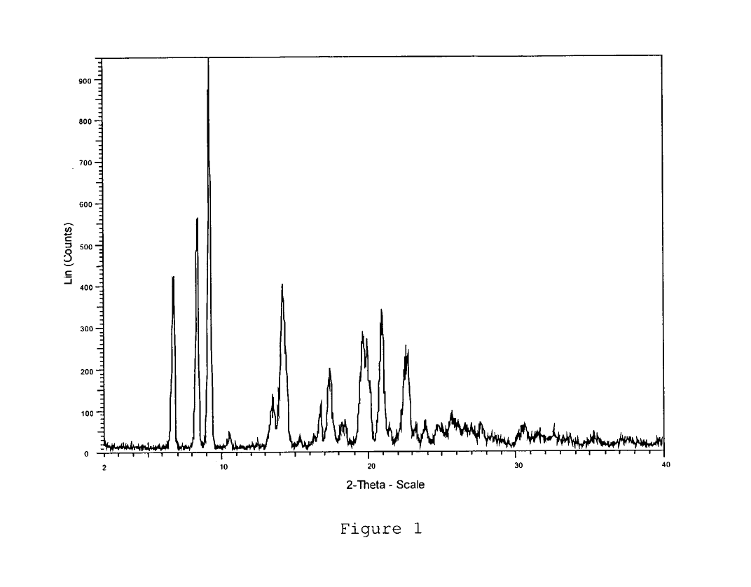

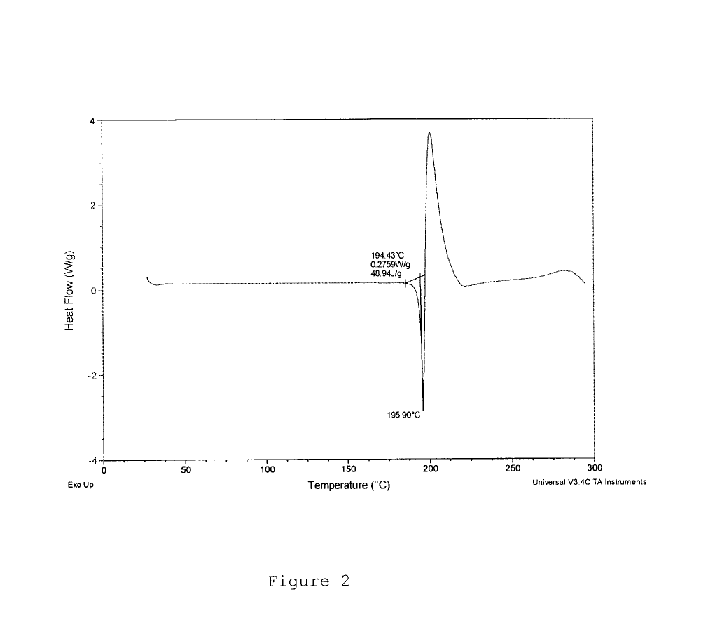

or a salt form thereof.

[13] In another preferred embodiment, the present invention provides a novel compound wherein the compound of formula III is a methanesulfonic acid salt.

[14] In another embodiment, the present invention provides a novel compound of formula IV:

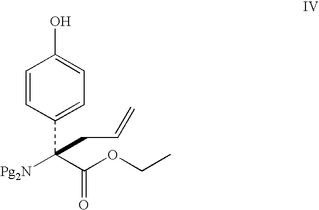

wherein P92 is an amino protecting group or represents H and an amino protecting group.

[15] In another preferred embodiment, the present invention provides a novel compound wherein Pg₂ is tert-butoxycarbonyl.

[16] In another embodiment, the present invention provides a novel compound of formula VIII or a salt form thereof:

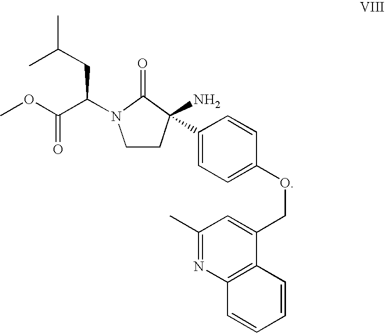

[17] In another preferred embodiment, the present invention provides a novel compound wherein the compound of formula VIII is a bis-methanesulfonic acid salt.

[18] In another embodiment, the present invention provides a novel crystalline, free-base form of Compound J ((2R)-2-((3R)-3-amino-3-{-[(2-methyl-4-quinolinyl)methoxy]phenyl}-2-oxopyrrolidinyl)-N-hydroxy-4-methylpentanamide):

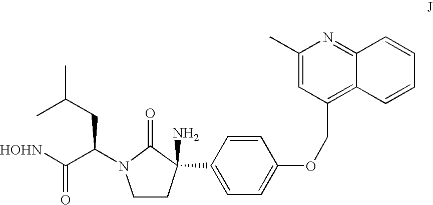

[19] In another preferred embodiment, the present invention provides a novel compound, wherein the compound is characterized by an x-ray powder diffraction pattern substantially in accordance with that shown in FIG. 1.

[20] In another preferred embodiment, the present invention provides a novel compound, wherein the compound is characterized by a differential scanning calorimetry thermogram substantially in accordance with that shown in FIG. 2.

[21] In another preferred embodiment, the present invention provides a novel compound, wherein the compound is characterized by a differential scanning calorimetry thermogram having a melt onset at about 194.4±0.5° C. with melting peak at about 195.9±0.5° C. followed by decomposition, wherein the DSC is operated at a rate of about 10° C./minute.

[22] In another preferred embodiment, the present invention provides a novel compound, wherein the compound is characterized by an x-ray powder diffraction pattern with its most intense reflections comprising the following 2θ values 6.7±0.2; 8.4±0.2; 9.2±02; 13.5±0.2; 14.2±0.2; 16.7±0.2; 17.4±0.2; 19.6±0.2; 19.9±0.2; 20.1±0.2; 20.9±0.2; and, 22.6±0.2 and a differential scanning calorimetry thermogram substantially in accordance with that shown in FIG. 2.

In another embodiment, the present invention provides a novel pharmaceutical composition, comprising: a pharmaceutically acceptable carrier and a therapeutically effective amount of a compound of the present invention or a pharmaceutically acceptable salt form thereof.

In another embodiment, the present invention provides a novel method for treating an inflammatory disorder, comprising: administering to a patient in need thereof a therapeutically effective amount of a compound of the present invention or a pharmaceutically acceptable salt form thereof.

In another embodiment, the present invention provides a novel method of treating a condition or disease mediated by MMPs, TACE, or a combination thereof in a mammal, comprising: administering to the mammal in need of such treatment a therapeutically effective amount of a compound of the present invention or a pharmaceutically acceptable salt form thereof.

In another embodiment, the present invention provides a novel method comprising: administering a compound of the present invention or a pharmaceutically acceptable salt form thereof in an amount effective to treat a condition or disease mediated by MMPs, TACE, or a combination thereof.

In another embodiment, the present invention provides a novel method of treating a disease or condition, wherein the disease or condition is selected from acute infection, acute phase response, age related macular degeneration, alcoholic liver disease, allergy, allergic asthma, anorexia, aneurism, aortic aneurism, asthma, atherosclerosis, atopic dermatitis, autoimmune disease, autoimmune hepatitis, Bechet's disease, cachexia, calcium pyrophosphate dihydrate deposition disease, cardiovascular effects, chronic fatigue syndrome, chronic obstruction pulmonary disease, coagulation, congestive heart failure, corneal ulceration, Crohn's disease, enteropathic arthropathy, Felty's syndrome, fever, fibromyalgia syndrome, fibrotic disease, gingivitis, glucocorticoid withdrawal syndrome, gout, graft versus host disease, hemorrhage, HIV infection, hyperoxic alveolar injury, infectious arthritis, inflammation, intermittent hydrarthrosis, Lyme disease, meningitis, multiple sclerosis, myasthenia gravis, mycobacterial infection, neovascular glaucoma, osteoarthritis, pelvic inflammatory disease, periodontitis, polymyositis/dermatomyositis, post-ischaemic reperfusion injury, post-radiation asthenia, psoriasis, psoriatic arthritis, pulmonary emphysema, pydoderma gangrenosum, relapsing polychondritis, Reiter's syndrome, rheumatic fever, rheumatoid arthritis, sarcoidosis, scleroderma, sepsis syndrome, Still's disease, shock, Sjogren's syndrome, skin inflammatory diseases, solid tumor growth and tumor invasion by secondary metastases, spondylitis, stroke, systemic lupus erythematosus, ulcerative colitis, uveitis, vasculitis, and Wegener's granulomatosis.

In another embodiment, the present invention provides a method for treating inflammatory disorders, comprising: administering, in combination, to a host in need thereof, a therapeutically effective amount of:

(a) one of the compounds of the present invention; and,

(b) one or more additional anti-inflammatory agents selected from selective COX-2 inhibitors, interleukin-1 antagonists, dihydroorotate synthase inhibitors, p38 MAP kinase inhibitors, TNF-α inhibitors, TNF-α sequestration agents, and methotrexate.

In another embodiment, the present invention provides novel compounds of the present invention for use in therapy.

In another embodiment, the present invention provides the use of novel compounds of the present invention for the manufacture of a medicament for the treatment of a condition or disease mediated by MMPs, TACE, or a combination thereof.

This invention also encompasses all combinations of preferred aspects of the invention noted herein. It is understood that any and all embodiments of the present invention may be taken in conjunction with any other embodiment to describe additional even more preferred embodiments of the present invention. It is also understood that each and every element of any embodiment is intended to be a separate specific embodiment. Furthermore, any elements of an embodiment are meant to be combined with any and all other elements from any of the embodiments to describe additional embodiments.

### Definitions

The present invention can be practiced on multigram scale, kilogram scale, multikilogram scale, or industrial scale. Multigram scale, as used herein, is preferable in the sale wherein at least one starting material is present in 10 grams or more, more preferable at least 50 grams or more, even more preferably at least 100 grams or more. Multikilogram scale, as used herein, is intended to mean the scale wherein more than one kilo of at least one starting material is used. Industrial scale as used herein is intended to mean a scale which is other than a laboratory sale and which is sufficient to supply product sufficient for either clinical tests or distribution to consumers.

As used herein, equivalents are intended to mean molar equivalents unless otherwise specified.

The reactions of the synthetic methods claimed herein are carried out in suitable solvents which may be readily selected by one of skill in the art of organic synthesis, the suitable solvents generally being any solvent which is substantially non-reactive with the starting materials (reactants), the intermediates, or products at the temperatures at which the reactions are carried out, i.e., temperatures which may range from the solvent's freezing temperature to the solvent's boiling temperature. A given reaction may be carried out in one solvent or a mixture of more than one solvent. Depending on the particular reaction step, suitable solvents for a particular reaction step may be selected.

Suitable polar solvents include, but are not limited to, ether and aprotic solvents.

Suitable ether solvents include: dimethoxymethane, tetrahydrofuran, 1,3-dioxane, 1,4-dioxane, furan, diethyl ether, 1,2-dimethoxyethane, diethoxymethane, dimethoxymethane, ethylene glycol dimethyl ether, ethylene glycol diethyl ether, diethylene glycol dimethyl ether, diethylene glycol diethyl ether, triethylene glycol dimethyl ether, and t-butyl methyl ether.

Suitable aprotic solvents may include, by way of example and without limitation, ether solvents, tetrahydrofuran (THF), dimethylformamide (DMF), 1,2-dimethoxyethane, diethoxymethane, dimethoxymethane, dimethylacetamide (DMAC), benzene, toluene, 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, and hexamethylphosphoramide.

Suitable hydrocarbon solvents include, but are not limited to, benzene, cyclohexane, pentane, hexane, hexanes, toluene, cycloheptane, methylcyclohexane, heptane, ethylbenzene, m-xylene, o-xylene, p-xylene, octane, indane, nonane, and naphthalene.

As used herein, an alcohol solvent is a hydroxy-substituted compound that is liquid at the desired temperature (e.g., room temperature). Examples of alcohols include, but are not limited to, methyl alcohol, ethyl alcohol, n-propyl alcohol, and i-propyl alcohol.

As used herein, the term “amino protecting group” (or “N-protected”) refers to any group known in the art of organic synthesis for the protection of amine groups. As used herein, the term “amino protecting group reagent” refers to any reagent known in the art of organic synthesis for the protection of amine groups that may be reacted with an amine to provide an amine protected with an amine-protecting group. Such amino protecting groups include those listed in Greene and Wuts, “Protective Groups in Organic Synthesis” John Wiley & Sons, New York (1991) and “The Peptides: Analysis, Synthesis, Biology, Vol. 3, Academic Press, New York, (1981), the disclosure of which is hereby incorporated by reference. Examples of amino protecting groups include, but are not limited to, the following: 1) acyl types such as formyl, trifluoroacetyl (TFA), phthalyl, and p-toluenesulfonyl; 2) aromatic carbamate types such as benzyloxycarbonyl (cbz) and substituted benzyloxycarbonyls, 2-(p-biphenyl)-1-methylethoxycarbonyl, and 9-fluorenylmethyloxycarbonyl (Fmoc); 3) aliphatic carbamate types such as tert-butyloxycarbonyl (Boc), ethoxycarbonyl, diisopropylmethoxycarbonyl, and allyloxycarbonyl; 4) cyclic alkyl carbamate types such as cyclopentyloxycarbonyl and adamantyloxycarbonyl; 5) alkyl types such as triphenylmethyl and benzyl; 6) trialkylsilane such as trimethylsilane; and 7) thiol containing types such as phenylthiocarbonyl and dithiasuccinoyl.

Amino protecting groups may include, but are not limited to the following: 2,7-di-t-butyl-[9-(10,10-dioxo-10,10,10,10-tetrahydrothio-xanthyl)]methyloxycarbonyl; 2-trimethylsilylethyloxycarbonyl; 2-phenylethyloxycarbonyl; 1,1-dimethyl-2,2-dibromoethyloxycarbonyl; 1-methyl-1-(4-biphenylyl)ethyloxycarbonyl; benzyloxycarbonyl; p-nitrobenzyloxycarbonyl; 2-(p-toluenesulfonyl)ethyloxycarbonyl; m-chloro-p-acyloxybenzyloxycarbonyl; 5-benzylsoxazolylmethyloxycrbonyl; p-(dihydroxyboryl)benzyloxycarbonyl; m-nitrophenyloxycarbonyl; o-nitrobenzyloxycarbonyl; 3,5-dimethoxybenzyloxycrbonyl; 3,4-dimethoxy-6-nitrobenzyloxycarbonyl; N′-p-toluenesulfonylaminocarbonyl; t-amyloxycarbonyl; p-decyloxybenzyloxycarbonyl; diisopropylmethyloxycarbonyl; 2,2-dimethoxycarbonylvinyloxycarbonyl; di(2-pyridyl)methyloxycarbonyl; 2-furanylmethyloxycarbonyl; phthalimide; dithiasuccinimide; 2,5-dimethylpyrrole; benzyl; 5-dibenzylsuberyl; triphenylmethyl; benzylidene; diphenylmethylene; and, methanesulfonamide.

As used herein, “strong base” or “strongly basic conditions” is intended to include, but not be limited to, alkyl lithiums, lithium amides, hydride bases, other organometallic bases, and t-butoxides. Examples of strong bases include, but are not limited to, lithium tert-butoxide, sodium tert-butoxide, potassium tert-butoxide, methyl lithium, ethyl lithium, n-propyl lithium, i-propyl lithium, n-butyl lithium, i-butyl lithium, s-butyl lithium, t-butyl lithium, hexyl lithium, lithium bis(trimethylsilyl)amide, lithium diisopropylamide, lithium 2,2,6,6-tetramethylpiperidine, potassium bis(trimethylsilyl)amide, potassium hydride, and sodium hydride.

As used herein, “substituted amine base” is intended to include, but not be limited to, trialkylamines wherein the three alkyl groups can be the same or different. Examples of alkyl include, but are not limited to, methyl, ethyl, n-propyl, i-propyl, n-butyl, s-butyl, t-butyl, n-pentyl, and s-pentyl. The alkyl groups on the substituted amine base also include cycloakyl groups (e.g., cyclopropyl, cyclobutyl, cyclopentyl, and cyclohexyl) and cycloalkyl-alkyl groups (e.g., cyclopropyl-methyl, cyclobutyl-methyl, cyclopentyl-methyl, and cyclohexyl-methyl). Examples of substituted amine bases include, but are not limited to, triimethylamine, triethylamine, tri-n-propylamine, and diisopropylethylamine.

As used herein, “borohydride reducing agent” is intended to include borohydride and borate reducing agents. These agents generally involve a cationic portion (e.g., sodium) and an anionic portion (e.g., borohydride). Examples of the cationic portion of the borohydride reducing agent include, but are not limited to, sodium, lithium, potassium, tetramethylammonium, tetrmethylammonium, tetrabutylammonium, cetyltrimethylammonium, benzyltriethylammonium, and methyltrioctylammonium. Examples of the anionic portion of the borohydride reducing agent include, but are not limited to, tri-sec-butylborohydride, trisiamylborohydride, triethylborohydride, triphenylborohydride, cyanoborohydride, triacetoxyborohydride, trimethoxyborohydride, triethoxyborohydride, and octahydrotriborate.

Preferably, the molecular weight of compounds of the present invention is less than about 500, 550, 600, 650, 700, 750, 800, 850, 900, 950, or 1000 grams per mole. More preferably, the molecular weight is less than about 950 grams per mole. Even more preferably, the molecular weight is less than about 850 grams per mole. Still more preferably, the molecular weight is less than about 750 grams per mole.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties.

The present invention is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

The present invention describes compounds in substantially pure form. “Substantially pure” as used herein is intended to mean at least 90, 91, 92, 93, 94, 95, 96, 97, 98, 99, to 100% pure.

For x-ray diffraction, the present invention is intended to encompass compounds yielding diffractograms that are “substantially in accordance” with those presently shown. A diffractogram “substantially in accordance” would be one that comprises 4, 5, 6, 7, 8, 9, 10, 12, 14, 16, 18, 20, 22, 24, 26, 28, 30, 32, 34, 36, 38, 40 or more of the peaks (i.e., 2θ values) within experimental error. Preferably, it would contain ten or more of the peaks. More preferably, it would contain twenty or more of the peaks. Even more preferably, it would contain thirty or more of the peaks. Alternatively, “substantially in accordance” is intended to mean a diffractogram having 20, 25, 30, 35, 40, 45, 50, 55, 60, 65, 70, 75, 80, 85, 90, 95% or more of the same peaks within experimental error. The relative intensities of the peaks may vary, depending upon the sample preparation technique, the sample mounting procedure and the particular instrument employed. Moreover, instrument variation and other factors may affect the 2θ values. Therefore, peak assignments inherently include experimental error and may vary by plus or minus 0.2.

For differential scanning calorimetry (DSC), it is known that the temperatures observed will depend upon the rate of temperature change as well as sample preparation technique and the particular instrument employed. Thus, the values shown in the thermograms may vary by plus or minus 4° C. A thermogram “substantially in accordance” would be one whose peaks vary by plus or minus 4° C.

When any variable (e.g., R⁶) occurs more than one time in any constituent or formula for a compound, its definition at each occurrence is independent of its definition at every other occurrence. Thus, for example, if a group is shown to be substituted with 0-2 R⁶, then said group may optionally be substituted with up to two R⁶ groups and R⁶ at each occurrence is selected independently from the definition of R⁶. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

“Halo” or “halogen” as used herein refers to fluoro, chloro, bromo, and iodo; and “counterion” is used to represent a small, negatively charged species such as chloride, bromide, hydroxide, acetate, and sulfate.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethyl alcohol, 2-propyl alcohol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a mammalian subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate and benzoate derivatives of alcohol and amine functional groups in the compounds of the present invention.

“Stable compound” and “stable structure” are meant to indicate a compound that is sufficiently robust to survive isolation to a useful degree of purity from a reaction mixture, and formulation into an efficacious therapeutic agent.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. When a substituent is keto (i.e., ═O) group, then 2 hydrogens on the atom are replaced.

As used herein, “treating” or “treatment” covers the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

“Therapeutically effective amount” is intended to include an amount of a compound of the present invention or an amount of the combination of compounds claimed effective to inhibit HIV infection or treat the symptoms of HIV infection in a host. The combination of compounds is preferably a synergistic combination. Synergy, as described for example by Chou and Talalay, Adv. Enzyme Regul. 22:27-55 (1984), occurs when the effect (in this case, inhibition of HIV replication) of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased antiviral effect, or some other beneficial effect of the combination compared with the individual components.

### Synthesis

By way of example and without limitation, the present invention may be further understood by the following schemes and descriptions. Scheme 1 exemplifies how a desired end product can be formed using the presently claimed process and intermediates.

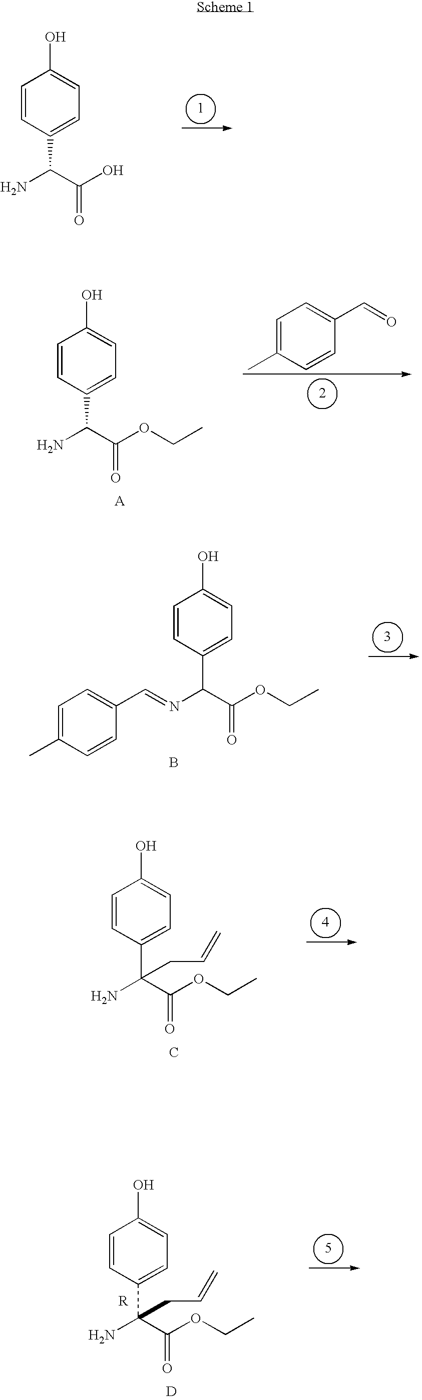

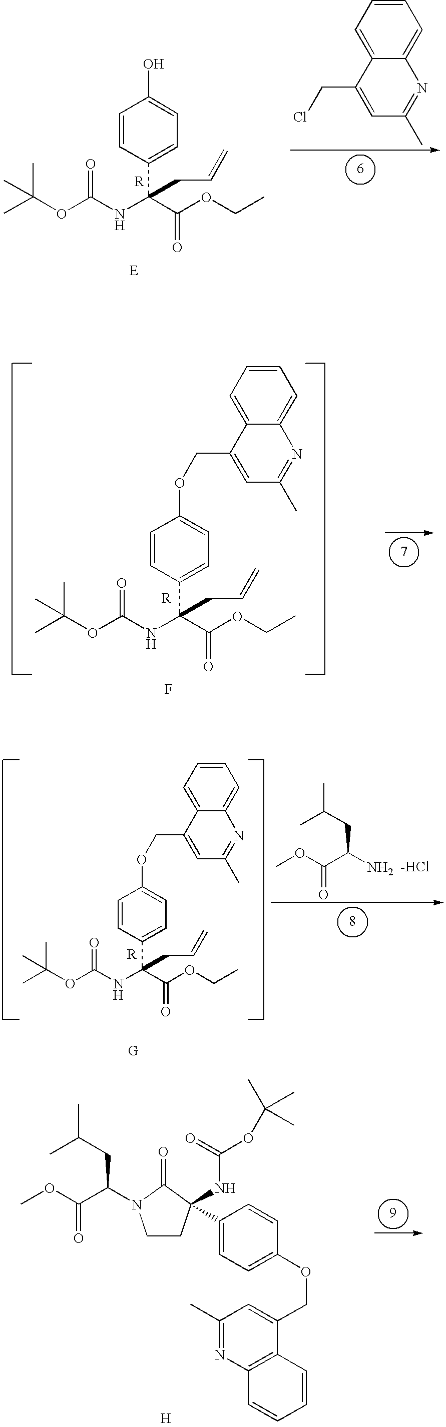

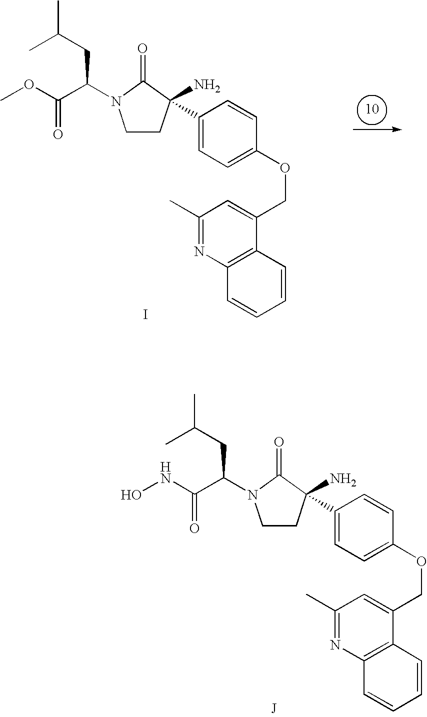

Reaction 1: Preparation of Compound A

Compound A can be formed by esterification of D-4-hydroxyphenylglycine. Preferably compound A is an ethyl ester (as shown). However, other esters (e.g., methyl) can also be formed. The ester can be made by contacting the starting glycine with an alcohol (e.g., methyl alcohol or ethyl alcohol) in the presence of an acid. A preferred alcohol is ethyl alcohol. Acids that can be used include, but are not limited to, methanesulfonic acid, sulfuric acid, phosphoric acid, p-toluenesulfonic acid, and benzenesulfonic acid. Preferably the acid is methanesulfonic or sulfuric. More preferably it is methanesulfonic. It is preferred to used about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.2, 3.4, 3.6, 3.8, 4, 4.2, 4.4, 4.6, 4.8, to 5 equivalents of acid, more preferably about 1.9. Generally, the desired alcohol is also the solvent for the esterification. If desired, the reaction can be heated up to the reflux point of the solvent.

Reaction 2: Preparation of Compound B

It is usually desired to protect the amine group of Compound A. This can be accomplished by forming a moiety like the 4-methylbenzylimine of Compound B. The amino protecting group is preferably formed from an aldehyde (e.g., benzaldehyde or a substituted benzaldehyde). Useful aldehydes include, but are not limited to, p-tolualdehyde, benzaldehyde, and 4-chloro-benzaldehyde. From about 1, 1.01, 1.02, 1.03, 1.04, 1.05, 1.06, 1.07, 1.08, 1.09, 1.1, 1.2, 1.3, 1.4, 1.5, 1.6, 1.7, 1.8, 1.9, to 2 equivalents of aldehyde are preferred. About 1.04 equivalents are more preferred. An aromatic solvent is preferred. Examples of aromatic solvents include, but are not limited to, toluene, xylene, and anisole. Preferably, the solvent is toluene. It is also preferred to use reaction conditions that remove water (e.g., distillation).

Reaction 3: Preparation of Compound C

Compound C is generally prepared by alkylation (i.e., allylation) and then deprotection of the glycine amine. Allyl bromide is a preferred alkylating agent. Preferably from 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, to 1.2 equivalents of alkylating agent are used, more preferably, 1.06 equivalents. The alkylation is conducted under strongly basic conditions. Preferred bases include lithium bis(trimethylsilyl)amide, lithium tert-butoxide, sodium tert-butoxide, and potassium tert-butoxide. The more preferred bases are lithium tert-butoxide and sodium tert-butoxide. An even more preferred base is lithium tert-butoxide. Preferably from 2, 2.1, 2.2, 2.3, 2.4, 2.5, 2.6, 2.7, 2.8, 2.9, to 3 equivalents of base are used, more preferably 2.1. An aprotic solvent is usually used for the alkylation reaction. Preferred aprotic solvents include tetrahydrofuran, diethoxymethane, dimethylformamide, and tert-butylmethylether. The more preferred solvents are tetrahydrofuran and diethoxymethane. An even more preferred solvent is tetrahydrofuran.

Compound B can undergo two competing alkylation reactions, oxygen-alkylation and carbon-alkylation. The preferred ratio of carbon- to oxygen-alkylation is from about 50, 100, 200, 300, 400, 500, 600, 700, 800, 900, to 1000 to 1 (i.e., 50-1000:1). It has been surprisingly found that the desired carbon-alkylation can be favored by careful selection of the strong base. In view of this, lithium tert-butoxide is an even more preferred base.

Once complete, the allylation reaction can be quenched, and the amino group can be deprotected by the addition of an acid. A preferred acid is aqueous hydrochloric acid.

Compound C is preferably isolated in salt form. A preferred salt form is the methanesulfonic acid salt. Salt formation is preferably run in the presence of a polar aprotic solvent and an alcohol, more preferably ethyl acetate and 2-propyl alcohol.

Reaction 4: Preparation of Compound D

After alkylation, Compound C can be converted to Compound D by enantiomeric resolution, preferably enzymatic resolution. Pig Liver Esterase (PLE) is a preferred enzyme for this resolution. From about 100, 110, 120, 130, 140, 150, 160, 170, 180, 190, to 200 KU/mole of PLE is preferred.

Additional components are usually helpful when running an enzymatic resolution. A base is preferably used. Bases for this reaction preferably include, but are not limited to, sodium hydroxide and potassium carbonate, with sodium hydroxide being more preferred. From about 0.9, 1, to 1.1 equivalents of based are preferred, 1 equivalent being more preferred. A non-ionic surfactant is preferably used. Non-ionic surfactants preferably include, but are not limited to, Antarox BL-225 and triton X-100®, with Antarox BL-225 being more preferred. From about 2, 2.5, 3, 3.5, 4, 4.5, 5 weight % of non-ionic surfactant is preferably used, 5 wt % being more preferred. A buffer is preferably used. Preferred buffers include, but are not limited to potassium phosphate di-basic and tris(hydroxylmethyl)amino-methane, with tris(hydroxylmethyl)amino-methane being more preferred. From about 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, to 0.3 equivalents of buffer are preferably used, 0.17 being more preferred. Solvents that can be used include, but are not limited to, water, water-acetonitrile, 2-propyl alcohol, and ethyl acetate. Preferred solvents are water and water-acetonitrile (5:1 v/v). A more preferred solvent is water.

The preferred pH of the enzymatic resolution will depend upon the enzyme selected. For PLE, it is preferred that the pH be from about 7.8, 7.9, 8.0, 8.1, to 8.2, with 8.0 being more preferred. The pH of the reaction can be adjusted by addition of an acid or base. Preferably, the components of the reaction are added in such a way as to form an acidic pH that can be slowly raised until the desired operating pH is achieved. The pH can be raised by the addition of a base (e.g., sodium hydroxide).

Compound D is preferably isolated in salt form. A preferred salt form is the methanesulfonic acid salt. Salt formation is preferably run in the presence of a polar aprotic solvent and an alcohol, more preferably ethyl acetate and 2-propyl alcohol. The enantiomeric excess (ee) obtained for Compound D is preferably about 90, 91, 92, 93, 94, 95, 96, 97, 98, to 99%, with greater than 96% being more preferred. It is even more preferred that the ee be greater than 98%.

Reaction 5: Preparation of Compound E

Compound E is formed by protecting the amino group of Compound D. A preferred protecting group is the tert-butoxycarbonyl group, though other amino protecting groups could be used. Preferably, Compound D is contacted with an amino protecting group reagent in the presence of a base. Preferred amino protecting group reagents include, but are not limited to, carbonates and carbamates. Preferred amino protecting group reagents include, but are not limited to, di-tert-butyl dicarbonate and 2-(tert-butoxycarbonyloxyimino)-2-phenylacetonitrile. A more preferred amino protecting group reagent is di-tert-butyl dicarbonate. From about 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, to 3 equivalents of amino protecting group reagent are preferably used, 1.95 equivalents being more preferred. A base is preferably used. Suitable bases include, but are not limited to, lithium carbonate, sodium carbonate, and potassium carbonate. Lithium carbonate is a more preferred base. From about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, to 4 equivalents of base are preferably used, 2.2 equivalents being more preferred. A polar, aprotic solvent is preferably used. Preferred polar aprotic solvents include, but are not limited to, ethyl acetate, tetrahydrofuran, isopropyl acetate, and dichloromethane. Ethyl acetate and tetrahydrofuran are more preferred solvents, with ethyl acetate being even more preferred. Water is also preferably used as a cosolvent.

Reaction 6: Preparation of Compound F

Compound F is prepared by alkylating the hydroxyl group of Compound E. The hydroxyl alkylating agent is selected based on the desired end product. It is preferably 4-chloromethyl-2-methylquinoline. From about 1, 1.05, 1.1, 1.15, to 1.2 equivalents of hydroxyl alkylating agent are preferably used, 1.15 being more preferred. A base is preferably used. Bases included, but are not limited to, potassium tert-butoxide and potassium carbonate. Potassium tert-butoxide is more preferred. Aprotic solvents are preferred for this reaction. Preferred aprotic solvents include, but are not limited to, tetrahydrofuran, diethoxymethane, and dimethylformamide. Tetrahydrofuran is a more preferred solvent. It is also preferred to use a phase transfer catalyst. A preferred phase transfer catalyst is tetrabutylammonium iodide. From about 0, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, to 1 equivalent of phase transfer catalyst is preferably present, 0.05 equivalents being more preferred. The alkylated product is preferably carried forward without purification.

Reaction 7: Preparation of Compound G

Cleaving the double bond of Compound F forms Compound G. A preferred cleaving method is to contact Compound F with ozone and then reduce the resulting product. Preferably about 0.9, 1, 1.1, 1.2, 1.3, 1.4, to 1.5 equivalents of ozone are used, 1 equivalent being more preferred. Preferred reducing agents include, but are not limited to, phosphines (e.g., triphenyl phosphine), phosphites (e.g., trimethyl phosphite), methyl sulfide, and zinc-acetic acid. Triphenyl phosphine is a more preferred reducing agent. From about 0.9, 0.95, 1, 1.05, 1.1, 1.15, to 1.2 equivalents of reducing agent are preferably used, 1.15 equivalents being more preferred. Preferred solvents include, but are not limited to, esters and hydrocarbons. More preferred solvents are ethyl acetate and dichloromethane, with ethyl acetate being even more preferred. The aldehyde product is preferably carried forward without purification.

Reaction 8: Preparation of Compound H

Reacting Compound G with an appropriate amino group (e.g., D-leucine methyl ester) forms the pyrrolidine ring of Compound H via a two step process of reductive amination and cyclization to a lactam. A preferred amino group reagent is D-leucine methyl ester hydrochloride salt. From about 1, 1.1, 1.2, 1.3, 1.4, to 1.5 equivalents of the amino group reagent are preferably used, 1.3 equivalents being more preferred. A substituted amine base (e.g., trialkylamine) is preferably used. Preferred substituted amine bases include, but are not limited to, triimethylamine, triethylamine, tri-n-propylamine, and diisopropylethylamine. Diisopropylethylamine is a more preferred base. From about 1, 1.1, 1.2, 1.3, 1.4, to 1.5 equivalents of base are preferably used, 1.3 equivalents being more preferred. Preferred solvents for the reductive amination include, but are not limited to, ethyl acetate, toluene, dichloromethane, and isopropyl acetate, ethyl acetate being more preferred.

For reductive amination, it is necessary to use a reducing agent. Numerous borohydride reducing agents are known (e.g., sodium borohydride, lithium borohydride, tetramethylammonium octahydrotriborate, and sodium triacetoxyborohydride) and are preferred, with sodium triacetoxyborohydride being more preferred. From about 1, 1.1, 1.2, 1.3, 1.4, 1.5, 1.6, 1.7, 1.8, 1.9, to 2 equivalents of reducing agent are preferably used, 1.5 equivalents being more preferred.

The reductive amination product is cyclized to the lactam by heating to eliminate ethyl alcohol in a solvent that includes, but is not limited to, ethyl acetate, toluene, isopropyl acetate, and 2-propyl alcohol.

Reaction 10: Preparation of Compound I

Amino-deprotection of Compound H provides Compound I. Deprotection is preferably done by contacting Compound H with an acid. Preferred acids include, but are not limited to, methanesulfonic acid, hydrochloric acid, sulfuric acid, benzenesulfonic acid, p-toluenesulfonic acid, and trifluoroacetic acid. Methanesulfonic acid is a more preferred acid. From about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, to 4 equivalents of acid are preferably used, 2.3 equivalents being more preferred. An alcoholic solvent is preferably used. Preferred alcoholic solvents are methyl alcohol, 2-propyl alcohol, ethyl alcohol, and n-propyl alcohol. More preferred alcoholic solvents are methyl alcohol and 2-propyl alcohol, with methyl alcohol being even more preferred.

Compound I is preferably isolated as a salt. Preferred salt forms include salts of the following acids: bis-methanesulfonic acid, mono-hydrochloric acid salt mono-hydrate, mono-sulfuric acid, bis-sulfuric acid, benzenesulfonic acid, p-toluenesulfonic acid, and trifluoroacetic acid. A more preferred salt is the bis-methanesulfonic acid salt.

Reaction 11: Preparation of Compound J

Conversion of the ester of Compound I to a hydroxamic acid group provides Compound J. A number of hydroxylamine reagents are known. Hydroxylamine reagents include, but are not limited to, hydroxylamine hydrochloride and hydroxylamine sulfate, with hydroxylamine hydrochloride being preferred. From about 1, 1.2, 1.4, 1.6, 1.8, 2, 2.2, 2.4, 2.6, 2.8, 3, 3.2, 3.4, 3.6, 3.8, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, to 10 equivalents of hydroxylamine reagent are preferred, with 4.7 equivalents being more preferred. An alcoholic solvent is preferred. Methyl alcohol and tertiary alchols such as t-amyl alcohol and t-butyl alcohol are more preferred. Methyl alcohol is even more preferred.

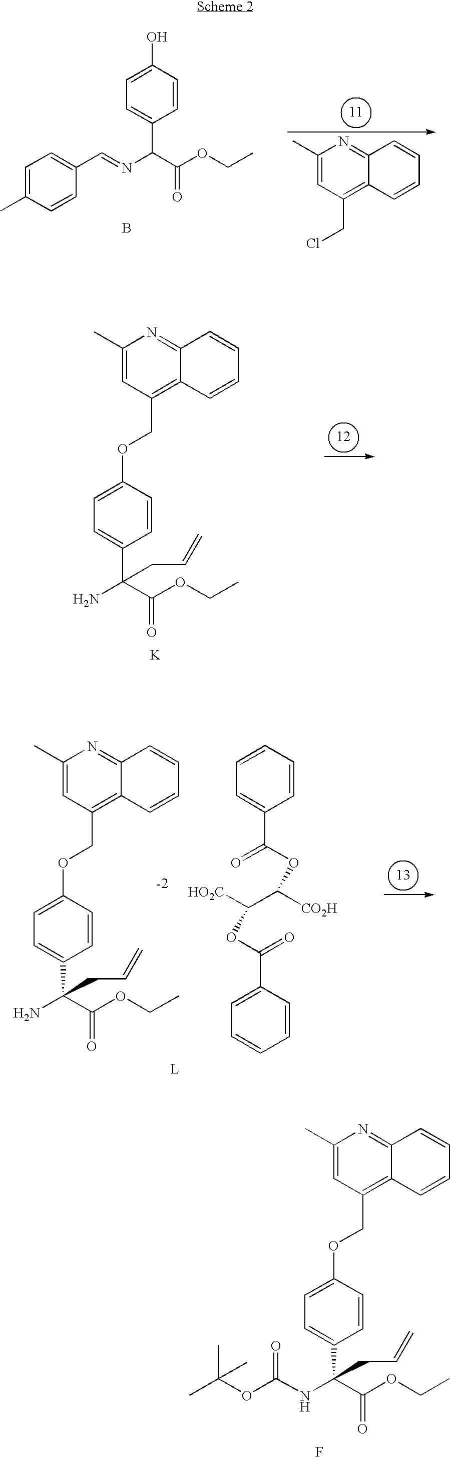

Reaction 11: Preparation of Compound K

Compound K is prepared by two selective sequential alkylations (i.e., carbon alkylation followed by oxygen alkylation) and then deprotection of the glycine amine. Allyl bromide is the preferred first alkylating reagent. Preferably from 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, to 1.2 equivalents of allyl bromide are used, more preferably, 1.06 equivalents. The alkylation is conducted under strongly basic conditions. Preferred bases include lithium tert-butoxide, sodium tert-butoxide, and potassium tert-butoxide. The more preferred bases are potassium tert-butoxide and sodium tert-butoxide. An even more preferred base is potassium tert-butoxide. Preferably from 2, 2.1, 2.2, 2.3, 2.4, 2.5, 2.6, 2.7, 2.8, 2.9, to 3 equivalents of base are used, more preferably 2.2. An aprotic solvent is usually used for the alkylation reaction. Preferred aprotic solvents include tetrahydrofuran, diethoxymethane, dimethylformamide, and tert-butylmethylether. The more preferred solvents are tetrahydrofuran and diethoxymethane. An even more preferred solvent is tetrahydrofuran.

Synthesis of Compound K is completed by alkylating the hydroxyl group. The hydroxyl alkylating agent is selected based on the desired end product. The hydroxyl alkylating agent is preferably 4-chloromethyl-2-methylquinoline. From about 1, 1.05, 1.1, 1.15, to 1.2 equivalents of hydroxyl alkylating agent are preferably used, 1 equivalent being more preferred. It is preferred to use a phase transfer catalyst. A preferred phase transfer catalyst is tetrabutylammonium iodide. From about 0, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, to 1 equivalent of phase transfer catalyst is preferably present, 0.05 equivalents being more preferred.

Once the second alkylation reaction is complete, it can be quenched by the addition of an acid. A preferred acid is aqueous hydrochloric acid. The quenching is preferably sufficient to deprotect the amino group and yield Compound K.

Reaction 12: Preparation of Compound L

Compound K can then be resolved into Compound L, its R-isomer. This resolution is preferably accomplished by successive treatments with a single enantiomer of a chiral acid. A preferred acid is di-benzoyl-L-tartaric acid. Preferably, Compound K is first contacted with from about 1.5, 1.6, 1.7, 1.8, 1.9, 2, 2.1, 2.2, 2.3, 2.4, 2.5, 2.6, 2.7, 2.8, 2.9, to 3 equivalents of acid, 1.7 equivalents being more preferred. Ethyl acetate is a preferred solvent for the first resolution. The solution of Compound K and acid is preferably seeded with the undesired enantiomer. The undesired salt can then be separated (e.g., filtered off) leaving an enatiomerically enriched solution of the R-isomer.

It is preferred to use a different, second solvent to complete the resolution. The first solvent is usually removed. Alcohilic solvents are preferred second solvents. Preferred alcoholic solvents include methyl alcohol, ethyl alcohol, and 2-propyl alcohol. More preferred alcoholic solvents are methyl alcohol and ethyl alcohol, with ethyl alcohol being even more preferred. A mixture of alcoholic solvents can also be used, e.g., 2-propyl alcohol/methyl alcohol. For the second resolution, the enatiomerically enriched Compound K is preferrably contacted with the same enantiomer of a chiral acid. A preferred acid is di-benzoyl-L-tartaric acid. For the second resolution, Compound K is preferably contacted with from about 1.5, 1.6, 1.7, 1.8, 1.9, 2, 2.1, 2.2, 2.3, 2.4, 2.5, 2.6, 2.7, 2.8, 2.9, to 3 equivalents of acid, 1.7 equivalents being more preferred. The second solution of Compound K and acid is preferably seeded with the desired enantiomer. The resulting solid is preferably collected and washed with an alcoholic solvent. The isolated salt can be recrystallized from alcohilic solvents. Preferred alcoholic solvents include methyl alcohol, ethyl alcohol, and 2-propyl alcohol or mixtures thereof. More preferred is a mixture of methyl alcohol and ethyl alcohol.

Compound K is preferably isolated as a salt. A preferred salt form is the salt of di-benzoyl-L-tartaric acid. The enantiomeric excess (ee) obtained for Compound K is preferably about 80, 81, 82, 83, 84, 85, 86, 87, 88, 89, 90, 91, 92, 93, 94, 95, 96, 97, 9, to 99%, with greater than 85% being more preferred. It is even more preferred that the ee be greater than 90%.

Reaction 13: Preparation of Compound F

Protecting the amino group of Compound K provides Compound F. This generally requires two reactions: (a) forming the free base and (b) reacting the amino group with an amino protecting reagent.

The free base of Compound K can be formed by methods known to those of ordinary skill in the art. A preferred method is to treat Compound K with a strong base in the presence of an aprotic solvent and water. Preferred strong bases include, but are not limited to, sodium hydroxide and potassium hydroxide. From about 2, 2.1, 2.2, 2.3, 2.4, 2.5, 2.6, 2.7, 2.8, 2.9, to 3 equivalents of strong base are preferably used, 2.2 equivalents being more preferred. An aprotic solvent is preferred for forming the free base. Ethyl acetate is a preferred aprotic solvent.

The amino group is preferably protected after the free base is made. A preferred protecting group is the tert-butyloxycarbonyl group, though other amino protecting groups can be used. Preferably, Compound D is contacted with an amino protecting group reagent in the presence of a base. Preferred amino protecting group reagents include, but are not limited to, carbonates and carbamates. Preferred amino protecting group reagents include, but are not limited to, di-tert-butyl dicarbonate and 2-(tert-butoxycarbonyloxyimino)-2-phenylacetonitrile. A more preferred amino protecting group reagent is di-tert-butyl dicarbonate. From about 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, to 3 equivalents of amino protecting group reagent are preferably used, 2 equivalents being more preferred. A base is preferably used. Suitable bases include, but are not limited to, tertiary amine bases and carbonate bases. Preferred bases include trimethylamine, triethylamine, tri-n-propylamine, diisopropylethylamine, lithium carbonate, sodium carbonate, and potassium carbonate. Diisopropylethylamine is a more preferred base. From about 1, 1.5, 2, 2.5, 3, 3.5, 4, 4.5, to 5 equivalents of base are preferably used, 1 equivalent being more preferred. A polar, aprotic solvent is preferably used. Preferred polar aprotic solvents include, but are not limited to, ethyl acetate, tetrahydrofuran, isopropyl acetate, dimethylformamide, and diethoxymethane. Diethoxymethane and tetrahydrofuran are more preferred solvents, with tetrahydrofuran being even more preferred. Water is also preferably used as a cosolvent.

Other features of the invention will become apparent in the course of the following descriptions of examplary embodiments which are given for illustration of the invention and are not intended to be limiting thereof.

### EXAMPLES

### Analytical Methods:

X-Ray Powder Diffraction:

X-ray powder diffraction data of the solid forms of Compound J were obtained with a Bruker AXS D8 Advance automated powder diffractometer. The diffractometer was equipped with a variable slit (q-compensating slit), a scintillation counter and a graphite monochromator. The radiation was CuKa (40 kV, 40 mA). Data were collected at room temperature from 2 to 40 degrees 2 theta with the sample spinning at 30 rpm; the step size was 0.02 degrees; the count time was 0.4 sec. per step. Samples were prepared on zero-background specimen holders as a thin layer of powdered material without solvent.

Differential Scanning Calorimetry (DSC):

The thermal properties of the crystalline form of Compound J was characterized with differential scanning calorimetry using a TA Instruments DSC 2920, with data analysis via a TA Instruments Universal Analysis. Samples were placed in sealed aluminum pans for analysis with an empty aluminum pan serving as the reference. Heating rates of 10° C. per minute was employed over a temperature range of 25° C. to 300° C. The instrument was calibrated with an indium standard.

Thermogravimetry (TGA):

The residual solvents in the crystalline form of Compound J were characterized with thermogravimetric analysis using a TA Instruments TGA 2950, with data analysis via a TA Instruments Universal Analysis. Samples were placed in open aluminum pans for analysis. Heating rates of 10° C. per minute was employed over a temperature range of 25° C. to 300° C. and corresponding weight loss in sample up to the melt of Compound J was recorded.

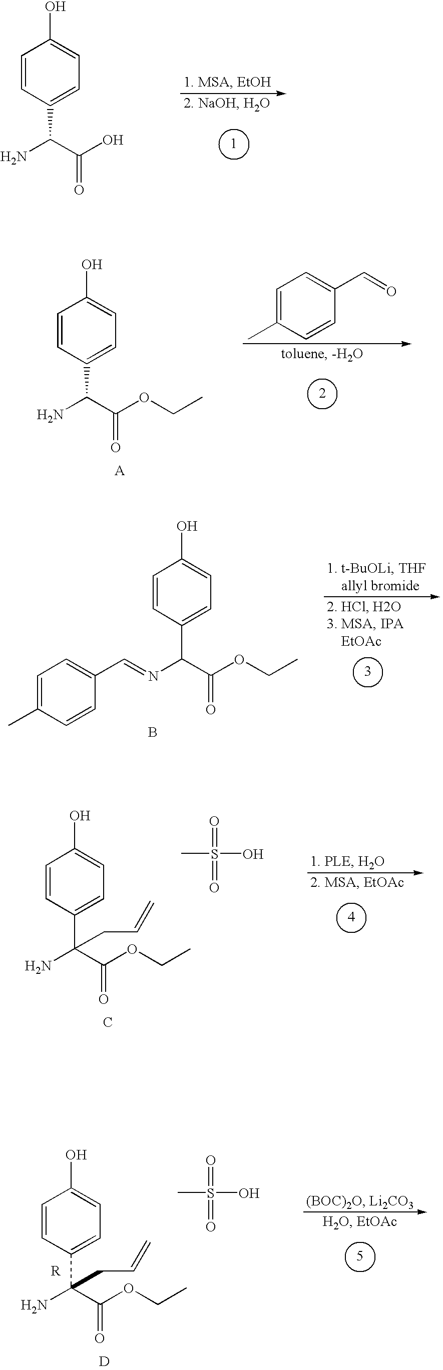

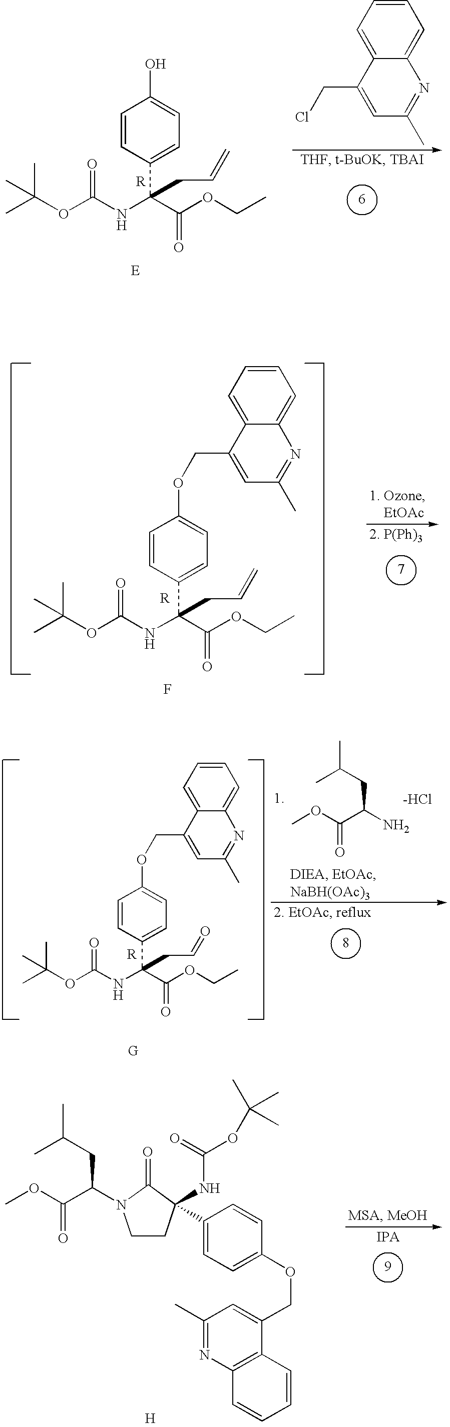

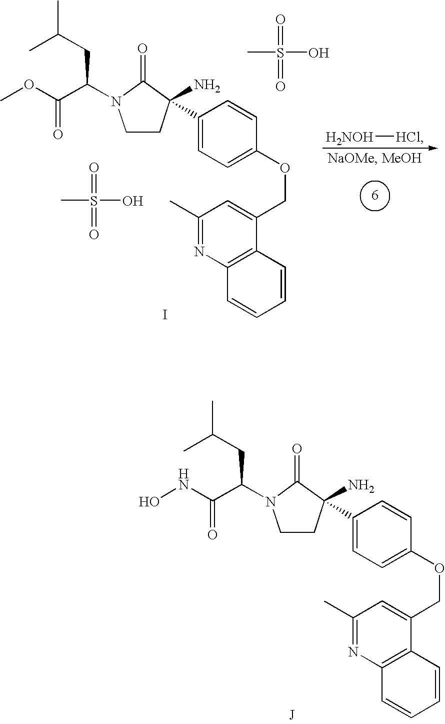

Preparation of Compound A

A 300-gallon reactor was charged with absolute ethyl alcohol (123 kg) and cooled to 10° C. D-4-hydroxyphenylglycine (78 kg, 0.47 kmol) was added, followed by methanesulfonic acid (86 kg, 0.89 kmol, 1.93 eq) at ≦78° C. The reaction mass was heated to 78° C. and aged for 2 h. The reactor was cooled to 55° C. and the reaction was determined complete by HPLC. The reactor was cooled to 40 ° C. and H₂O (468 L) was added. The reactor was cooled to 5° C. and 17% aqueous sodium hydroxide (prepared from 142 L H₂O, 71.3 kg 50% sodium hydroxide {0.89 kmol, 1.93 eq}) was slowly added over 2 h to provide a slurry of Compound A at pH=7.0 to 7.5. The slurry was stirred at 5° C. for 1 h. Compound A was isolated by centrifugation and washed with H₂O (3×106 kg). Compound A was vacuum dried at 50 to 55° C. to a constant weight. Compound A, 83.5 kg (92% yield), was isolated as a white to light yellow crystalline solid. ¹H NMR (400 MHz, DMSO-d₆) δ 7.18 (2H, d, J=8.5 Hz), 6.73 (2H, d, J=8.6 Hz), 4.40 (1H, s), 4.15-3.95 (2H, m), 1.12 (3H, t, J=7.1 Hz). ¹³C NMR (100 MHz, DMSO-d₆) δ 174.7, 157.1, 131.7, 128.2, 115.4, 60.6, 58.0, 14.3. Analysis Calculated for C₁₀H₁₃NO₃: C, 61.53; H, 6.71; N, 7.18. Found: C, 61.54; H, 6.57; N, 7.11.

Preparation of Compound B

A 300 gallon reactor was charged with toluene (685 kg) and cooled to 0° C. Compound A (80 kg, 0.41 kmol) was added followed by p-tolualdehyde (51.4 kg, 0.43 kmol, 1.04 eq) with a toluene (7 kg) flush. The reaction mass was distilled (90 to 115° C.) until approximately 180 kg distillate was collected. Fresh toluene, equivalent in mass to the distillate was added. The reaction mass was cooled to 85° C. and aged for 1 h, then cooled to 20° C. over 1 h and aged for 2 h. Compound B was isolated by centrifugation and washed with a toluene (200 kg)-heptane (157 kg) mixture in three portions. Compound B was vacuum dried at 50 to 55° C. to a constant weight. Compound B, 118 kg (97% yield) was isolated as a white to light yellow crystalline solid. ¹H NMR (400 MHz, CDCl₃) δ 8.25 (1H, s), 7.67 (2H, d, J=8.1 Hz), 7.26 (2H, d, J=8.6 Hz), 7.16 (2H, d, J=8.0 Hz), 6.76 (2H, d, J=8.6 Hz), 5.13 (1H, s), 4.25-4.11 (2H, m), 2.33 (3H, s), 1.20 (3H, t, J=7.1 Hz). ¹³C NMR (100 MHz, CDCl₃) δ 172.0, 164.0, 156.2, 141.8, 132.7, 129.3, 129.2, 129.1, 128.8, 115.9, 75.7, 61.6, 21.5, 14.0.

Preparation of Compound C

A 300-gallon glass lined reactor was charged with tetrahydrofuran (215.5 kg, THF) and cooled to −5° C. Compound B (62.0 kg, 0.21 kmol) was added, followed by allyl bromide (26.7 kg, 0.22 kmol, 1.06 eq) with a THF (2.5 kg) chase. The reactor was cooled to −5° C. and charged with 2 M lithium tert-butoxide in THF (192.6 kg, 0.44 kmol, 2.1 eq) over 1 h at ≦5° C. The reaction mass was sampled for conversion after 30 minutes and determined complete by HPLC. The reaction was quenched by adding 2 M aqueous hydrochloric acid (326 kg, 0.625 kmol, 3 eq), pH=1 after quench. Heptanes (107 kg) were added and the layers were separated, retaining the product rich aqueous phase. The pH of the aqueous phase was adjusted to 8-9 by adding a 16% aqueous sodium hydroxide solution (prepared by combining 32 kg 50% aqueous sodium hydroxide {0.40 kmol, 1.9 eq} and 62 kg H₂O). Sodium chloride (31 kg) and ethyl acetate (369 kg) were added and the layers were separated, retaining the product rich organic phase. Solvent exchanged to ethyl acetate to a final volume of −250 L by distillation at atmospheric pressure. Ethyl acetate was added to adjust the final volume. Cooled to 60° C. and added 2-propyl alcohol (9.6 kg). Cooled to 50° C. and added methanesulfonic acid (20.1 kg, 0.21 kmol, 1 eq). Cooled to 20° C. over 2 h. Cooled to 0° C. and aged for 1 h. Compound C was isolated by centrifugation and washed with ethyl acetate (2×56 kg) Compound C was vacuum dried at 50 to 55° C. to a constant weight. Compound C, 57 kg (82% yield) was isolated as a white to light yellow crystalline solid. ¹H NMR (400 MHz, DMSO d₆) δ 9.90 (1H, s), 8.83 (3H, s), 7.29 (2H, d, J=8.8 Hz), 6.85 (2H, d, J=8.8 Hz), 5.83-5.71 (1H, m), 5.30 (1H, d, J=17.0 Hz), 5.24 (1H, d, J=10.1 Hz), 4.30-4.15 (2H, m), 2.97 (2H, dd, J=6.8, 14.5 Hz), 2.35 (3H, s), 1.18 (3H t, J=7.1 Hz). ¹³C NMR (100 MHz, CD₃OD) δ 171.4, 160.4, 131.1, 128.9, 127.1, 123.7, 117.4, 66.1, 64.7, 41.5, 40.0, 14.7.

Preparation of Compound D

Charged 120 L of water to a 100-gallon reactor. Heated to 40° C. Charged 12 kg of Compound C. Stirred at moderate rate to dissolve the mixture. Charged 0.73 kg of Tris(hydroxymethyl)aminomethane. Charged 0.6 kg of Antarox BL-225. Carefully added 5 kg of NaOH (6 N, prepared earlier) while monitoring pH. The final pH was in the range of 6.8 to 7.5. Added NaOH (6 N) in small increments to adjust final pH to a range of 7.8 to 8.1. If pH was over 8.1, added HCl (6 N) to bring pH back to 8.0. Charged 0.27 kg PLE. Agitated the mixture at 150 rpm at 40° C. and pH=8.0±0.3 (NaOH (6 N) used for pH adjustment) for 5 h, at which time the reaction was judged complete by HPLC analysis. Cooled the reaction mass to 20° C. Charged 4 L of water. Charged 108 kg of ethyl acetate. Charged 18 kg of Celite®-560 and filtered through a Dacron cloth on a Nutsche filter. Rinsed the reactor with 54 kg of ethylacetate and used the rinse to wash the filter-cake. Combined the wash with filtrate. Rinsed the reactor with 40 L of water and used the rinse to wash the filter-cake. Combined the wash with filtrate. Charged the combined filtrate/washes to the reactor and separated the layers, retaining both phases. Charged the aqueous layer back to the reactor and back extracted with 54 kg of ethyl acetate. Charged both the organic phases to the reactor. Washed combined organic layers with 40 kg of saturated aqueous sodium chloride solution. Separated aqueous (lower) layer. Distilled off ethyl acetate and residual water to a constant b.p. (77° C.±1° C.). Cooled the batch to ˜50° C. Charged 2 kg of 2-propyl alcohol. Charged 1.8 kg of methanesulfonic acid in 4 equal portions of ˜0.45 kg over a period of 20 min. maintaining the temperature between 50 to 55° C. Cooled the batch to 25° C. over ˜100 min. Aged the slurry of crystalline Compound D MSA salt at 25° C. for 2 h. Cooled to 10° C. over ˜1 h. Filtered the slurry at 10° C. Rinsed the reactor with 12 kg of ethyl acetate twice and used the rinses to wash the filter-cake. Dried the product under vacuum at 50 to 55° C. with nitrogen purge to constant weight, 5 kg (42% yield). ¹H NMR (400 MHz, DMSO d₆) δ 9.90 (1H, s), 8.83 (3H, s), 7.29 (2H, d, J=8.8 Hz), 6.85 (2H, d, J=8.8 Hz), 5.83-5.71 (1H, m), 5.30 (1H, d, J=17.0 Hz), 5.24 (1H, d, J=10.1 Hz), 4.30-4.15 (2H, m), 2.97 (2H, dd, J=6.8, 14.5 Hz), 2.35 (3H, s), 1.18 (3H t, J=7.1 Hz). ¹³C NMR (100 MHz, CD₃OD) δ 171.4, 160.4, 131.1, 128.9, 127.1, 123.7, 117.4, 66.1, 64.7, 41.5, 40.0, 14.7. Analysis Calculated for C₁₄H₂₁NO₆S: C, 50.74; H, 6.39; N, 4.23; S, 9.68. Found: C, 50.43; H, 6.06; N, 4.08; S, 9.74.

Preparation of Compound E

A 300 gallon glass lined reactor was charged sequentially with Compound D (55 kg, 0.17 kmol), lithium carbonate (26.8 kg, 0.36 kmol, 2.2 eq), ethyl acetate (150 kg), di-tert-butyl dicarbonate ((BOC)₂O, 72.4 kg, 0.33 kmol, 1.95 eq), and water (269 L). Heated to 40±2° C. and aged for 14 h. The reaction was sampled and determined complete by HPLC. Cooled the reaction mass to 20° C. and slowly charged acetic acid (32.9 kg, 0.55 kmol, 3.2 eq). Checked the pH, adjusted the pH to <6.5 with acetic acid. Separated the phases, retained the product rich organic phase. Washed the organic phase with water (269 L). Solvent exchanged to heptane to a final volume of 400 L by distillation at 100 mm Hg. The resulting slurry was cooled to 20° C. Compound E was isolated by filtration on a Nutsche filter and washed with heptane (2×75 kg). Compound E was vacuum dried at 50 to 55° C. to a constant weight. Compound E, 43 kg (78% yield) was isolated as a white to light yellow crystalline solid. ¹H NMR (400 MHz, CDCl₃) δ 7.21 (2H, d, J=8.7 Hz), 6.57 (2H, d, J=7.7 Hz), 6.19 (1H, s), 5.75-5.58 (1H, m), 5.18 (d, 1H, J=14.0 Hz), 5.14 (d, 1H, J=8.2), 4.25-4.00 (2H, m), 3.48-3.30 (1H, m), 3.25-3.10 (1H, m), 1.44 (9H, s br), 1.16 (3H, t, J=14.2 Hz). ¹³C NMR (100 MHz, CDCl₃) δ 172.5, 155.7, 154.0, 132.5, 130.6, 127.0, 119.4, 115.5, 79.9, 64.4, 62.2, 38.1, 28.4, 14.0. Analysis Calculated for C₁₈H₂₅NO₅: C, 64.46; H, 7.51; N, 4.18. Found: C, 60.03; H, 7.38; N, 4.14.

Preparation of Compound F

A reactor was charged with 120 kg compound E (0.36 kmol) and 532 kg THF and cooled to 5° C. A solution of 48.2 kg potassium t-butoxide (0.43 kmol, 1.20 eq.) and 150 kg THF was added while maintaining the temperature below 10° C., with a chase of 32 kg THF. Aged at 0 to 10° C. for 15 minutes then added 68.5 kg 4-chloromethyl-2-methylquinoline (AB9871, 0.36 kmol, 1.00 eq) and 6.6 kg tetrabutylammonium iodide (0.018 kmol, 0.05 eq). Heated to 35 to 40° C. for 3 h at which time the reaction was judged complete by HPLC. Cooled to ˜20° C. Added 725 L water, 18.2 kg acetic acid (0.30 kmol, 0.85 eq) and 621 kg ethyl acetate and separated the phases. Washed the organic phase with a solution of 98 kg citric acid (0.51 kmol, 1.43 eq) and 900 L water. Washed the organic phase with a solution of 72 kg sodium chloride and 650 L water. To the organic phase added 690 L water and adjusted to pH 7.1 by addition of 33 L of 33 wt % aqueous NaOH. After the pH adjustment, the layers were separated and the aqueous layer was discarded. Washed the organic phase with a solution of 72 kg sodium chloride and 650 L water. Vacuum-distilled to ˜600 L. Portion-wise added an additional 1200 L ethyl acetate while continuing to vacuum distill at ˜600 to 900 L volume to provide a solution of compound F in ethyl acetate. The resulting ethyl acetate solution of compound F was carried forward directly to the next processing step.

Preparation of Compound G

Added 1460 L ethyl acetate to the previously prepared solution of compound F. The reaction mass was cooled to −65 to −70° C. Ozone was introduced subsurface to the reactor for 3.5 h HPLC until the reaction was judged complete by HPLC. The solution was purged of excess ozone and oxygen with nitrogen. A 5° C. solution of 113 kg triphenylphosphine (0.43 kmol, 1.20 eq) and 450 kg ethyl acetate was added over −10 minutes to the reaction mass with a 90 kg ethyl acetate chase. The reaction mass was warmed to 0° C. over 4 h. After 12 h at 0° C. the reaction mass was warmed to 12° C. and determined to be complete by HPLC. The reaction mass was washed with a solution of 13 kg sodium chloride in 980 L water. The resulting ethyl acetate solution of compound G was carried forward directly to the next processing step. The characterization data is from a sample of compound G purified by column chromatography. ¹H NMR (400 MHz, CDCl₃) δ 9.75 (1H, s), 8.09 (1H, d, J=8.4 Hz), 7.90 (1H, d, J=8.4 Hz), 7.71 (1H, dd, J₁=8.3, J₂=8.3, Hz), 7.53 (1H, dd, J₁=8.3, J₂=8.3 Hz), 7.43 (1H, s), 7.38 (2H, d, J=9.2 Hz), 7.01 (2H, d, J=9.2 Hz), 6.18 (1H, s), 5.47 (2H, s), 4.25-4.09 (3H, m), 3.58 (1H, d, J=17.6 Hz), 2.74 (1H, s), 1.38 (9H, s), 1.18 (3H, t). ¹³C NMR (100 MHz, CD₃OD) δ 199.3, 171.4, 158.9, 158.1, 154.0, 147.7, 141.8, 129.5, 129.3, 126.9, 126.1, 124.1, 122.7, 120.3, 115.0, 66.8, 62.5, 61.2, 28.2, 25.3, 13.8. MS (EI+): m/z: 493.2 (M+1), 437.1, 376.12, 350.1.

Preparation of Compound H

To the previously prepared solution of compound G in ethyl acetate was added 60.1 kg diisopropylethylamine (0.47 kmol, 1.3 eq) and 84.5 kg D-leucine methyl ester hydrochloride (0.47 kmol, 1.3 eq). The batch was cooled to 0 to 5° C. and held for 1 h. Portionwise added 110 kg sodium triacetoxyborohydride (0.52 kmol, 1.45 eq) at 0 to 5° C. Aged at 0±5° C. for 2 h at which point the reaction was judged complete by HPLC. Added 2000 L water and separated the phases. Added 150 kg toluene to the organic phase and extracted the organic phase with four portions (1000 L, 720 L, 720 L, 720 L) of an aqueous citric acid solution. Extracted a fifth time with a solution of 222 kg citric acid in 580 L water. The aqueous citric acid extracts were combined and washed with a mixture of 788 L ethyl acetate and 74 kg toluene. Added 550 L ethyl acetate and cooled the aqueous phase to 10 to 15° C. Adjusted the pH to 4.6 by addition of 866 L 30% aqueous sodium hydroxide while maintaining the batch temperature at <25° C. Separated the layers and back-extracted the aqueous phase with 790 L ethyl acetate. The two ethyl acetate phases were combined and washed with 500 L water to provide an ethyl acetate solution of a product of the above reductive amination of compound G. The ethyl acetate solution was distilled at atmospheric pressure to ˜800 L final volume (FIO: KF=0.166%). 400 L ethyl acetate was added and the batch again distilled to ˜800 L final volume. The reaction mass was held at reflux for 13 h and the cyclization to the lactam compound H was judged complete by HPLC analysis. Solvent exchanged to 2-propyl alcohol by atmospheric distillation at a volume of 600 to 700 L by portionwise addition of 2300 L 2-propyl alcohol at atmospheric pressure. Distilled to a final volume of ˜350 L. Cooled to 0 to 5° C. and seeded with 0.5 kg compound H. The crystallization was aged for 24 h at 0 to 5° C. The slurry was filtered on a centrifuge and the cake was washed with 250 L of 2-propyl alcohol. The product was dried at 40° C. for 8 h to afford 122 kg compound H, 59% overall yield from 120 kg compound E was obtained. ¹H NMR (400 MHz, CDCl₃) δ 8.09 (1H, d, J=8.4 Hz), 7.91 (1H, d, J=8.4 Hz), 7.72 (1H, dd, J₁=8.3, J₂=8.3, Hz), 7.53 (1H, dd, J₁=8.3, J₂=8.3, Hz), 7.48 (1H, s), 7.46 (2H, d, J=9.2 Hz), 7.00 (2H, d, J=9.2 Hz), 5.64 (1H, s), 5.49 (2H, s), 4.93 (1H, s, Br), 3.58 (3H, s), 3.40 (2H, m), 2.89 (1H, t), 2.77 (1H, s), 2.76 (3H, s), 1.81-1.72 (2H, m), 1.58 (1H, m), 1.42 (9H, s), 0.97 (6H, m). ¹³C NMR (100 MHz, CD₃OD) δ 173.7, 171.4, 158.9, 157.9, 154.7, 147.7, 142.0, 133.2, 129.4, 127.3, 126.1, 124.1, 122.6, 120.3, 114.9, 66.8, 63.2, 52.4, 52.1, 40.5, 36.9, 28.3, 25.4, 24.6, 23.3, 21.1. MS (DCI/NH₃): m/z: 576.3 (M+1), 520.2, 459.1. Analysis Calculated for C₃₃H₄₁N₃O₆: C, 68.85; H, 7.18; N, 7.30. Found: C, 68.69; H, 7.06; N, 7.14.

Preparation of Compound I

To a 200 gallon glass-lined reactor was charged 30.2 kg Compound H (0.052 kmol) and 90 kg methyl alcohol. Added 5.8 kg methanesulfonic acid (MSA, 0.06 kmol, 1.15 eq) gradually at 25° C. The batch was heated slowly to 55° C. Added another 5.8 kg methanesulfonic acid (0.06 kmol, 1.15 eq) gradually over 30 min at 55° C. The reactor contents were aged at 55° C. for 2 h and sampled for reaction completion (HPLC criterion: <0.5 A % Compound H). Added 214 L 2-propyl alcohol at 55° C. Cooled to 15° C. and aged at 15° C. for 1 h. Isolated on a centrifuge and washed the cake with 2-propyl alcohol (3×50 kg). Dried under vacuum at 50° C. to provide 31.7 kg Compound 190% yield. ¹H NMR (400 MHz, DMSO d₆) δ 9.07 (4H, s), 8.51 (1H, d, J=8.4 Hz), 8.32 (1H, d, J=8.6 Hz), 8.14 (1H, s), 8.14 (1H, t, J=8.6 Hz), 7.96 (1H, t, J=8.6 Hz), 7.61 (d, 2H, J=8.9 Hz), 7.39 (d, 2H, J=9.0 Hz), 5.94 (s, 2H), 4.80 (dd, 1H, J₁=4.0 Hz, J₂=11.3 Hz), 3.62 (s, 3H), 3.51 (1H, t, J=9.5 Hz), 3.15-3.28 (1H, m), 3.01 (3H, s,), 2.65-2.75 (1H, m), 2.48-2.60 (2H, m), 2.43 (6H, s), 1.85 (1H, t, J=12.2 Hz), 1.52-1.71 (2H, m), 0.96 (3H, d, J=6.3 Hz), 0.93 (3H, d, J=6.10 Hz). ¹³C NMR (100 MHz, CDCl₃) δ 172.9, 172.5, 160.6, 159.7, 156.5, 139.1, 136.1, 131.2, 130.0, 128.9, 126.3, 126.0, 122.1, 117.2, 67.8, 64.3, 53.4, 50.1, 41.5, 40.1, 38.1, 33.8, 26.2, 24.0, 22.0, 21.5. Analysis Calculated for C₃₀H₄₁N₃O₁₀S₂: C, 53.96; H, 6.19; N, 6.29; S, 9.60. Found: C, 53.83; H, 5.97; N, 6.15; S, 9.76.

Preparation of Compound J:

To a 30 gallon glass-lined reactor were charged 10 kg of hydroxylamine hydrochloride (0.14 kmol, 4.7 eq) and 15.6 kg methyl alcohol. The batch temperature was set to 50° C. and 64.6 kg of a 25 wt % solution of sodium methoxide in methyl alcohol was charged (sodium methoxide: 16.15 kg, 0.3 kmol, 10 eq) followed by a methyl alcohol rinse of the charging line. The reactor contents were heated to 55° C. and aged for 15 min. The batch was then cooled to 25° C. and filtered through a 36″ nutsch filter using a polypropylene filter bag. The filtrate was collected in a 100-gallon glass-lined reactor and cooled to 10° C. Compound I (20 kg, 0.03 kmol) was added and the batch was warmed to 25° C. for aged for 1 h. The reactor contents were sampled for reaction completion (HPLC criterion: >99.5 A % compound J). Once the reaction was deemed complete, ˜55 kg of 2N HCl solution (prepared using 126 kg purified water and 30 kg of concentrated HCl) was added and the reaction mass was sampled for pH measurement (acceptance criterion: pH ˜7.0). The batch was vacuum-distilled at ˜35° C. to remove ˜25 L methyl alcohol. The batch was then heated to 50° C. and a 1 L slurry of compound J (150 g) 1:4 methyl alcohol/water (volume ratio) was added. Water (92 L) was added uniformLy over 1 h at 50° C. to induce crystallization. The batch was cooled to 5° C. over a period of 2 h. The contents were filtered and the product washed first with a mixture of methyl alcohol/water (1:4 volume ratio) and then with pure water. The wet cake (˜20 kg) was analyzed to determine the weight % of water and charged to a clean 100-gallon reactor. Isopropyl alcohol (34 kg) was added and the batch was heated to 55° C. Once all the solids were dissolved, water was added to adjust the volume ratio to ˜55% water, 45% isopropyl alcohol. At 55° C., 2 L slurry of milled Compound J seeds (˜700 g) in 1:4 isopropyl alcohol/water (volume ratio) was charged. Purified water (67 kg) was charged through a cartridge filter gradually over a period of 3 h. The batch was cooled from 55° C. to 20° C. in 2 h, aged for 30 min and filtered through a 36″ nutsch filter using a Dacron® filter bag. The filter-cake was washed three times with a mixture of isopropyl alcohol-water (lst wash: 38 kg water, 8 kg isopropyl alcohol; 2^{nd} and 3^{rd} washes: 19 kg water, 4 kg isopropyl alcohol). The product was dried in a tray dryer under vacuum at 50° C. to provide 12.0 kg of Compound J in 84% yield.

FIG. 1 shows the characteristic XRPD pattern of the anhydrous form of Compound J. The crystalline form has characteristic XRPD reflections at the following 2Θ values: 6.7±0.2; 8.4±0.2; 9.2±0.2; 10.5±0.2; 13.5±0.2; 14.2±0.2; 15.3±0.2; 16.3±0.2; 16.7±0.2; 17.4±0.2; 18.1±0.2; 18.4±0.2; 19.6±0.2; 19.9±0.2; 20.1±0.2; 20.9±0.2; 21.4±0.2; 22.6±0.2; 23.2±0.2; 23.9±0.2; 24.8±0.2; 25.7±0.2; 27.6±0.2; 30.5±0.2; 32.6±0.2; 39.8±0.2.

FIG. 2 shows the differential scanning calorimetry thermogram of Compound J. The crystalline form of Compound J has a melt onset of 194.4±0.5° C. with melting peak at 195.9±0.5° C. followed by decomposition.

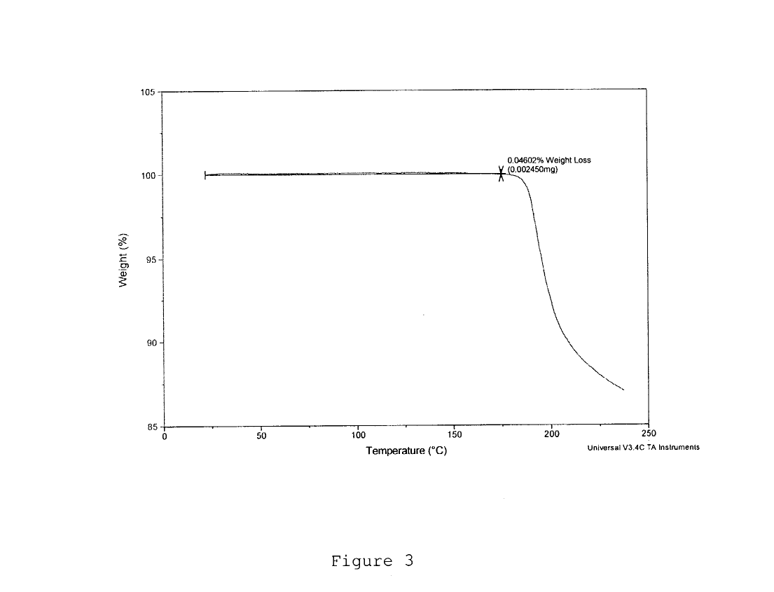

FIG. 3 shows the thermogravimetric analysis of Form T of Compound J. There is no significant weight loss up to the melt of the drug substance indicating that the crystalline drug substance is unsolvated.

The free-base form of Compound J is considered advantageous over the bis-hydrochloric acid salt, bis-methanesulfonic acid salt, and bis-triflouroacetic acid salt forms. Neither the bis-hydrochloric acid nor the bis-methanesulfonic acid salt form existed as a stable anhydrous form. The bis-hydrochloric acid and the bis-methanesulfonic acid salts were tested over a range of 10-90% relative humidity and exhibited weight gains of 6.9% and 1.2%. Both of these weight gains are undesirable. The free-base form is considered advantageous over the bis-triflouroacetic acid salt, since the bis-TFA salt would likely be toxic if administered on a chronic basis.

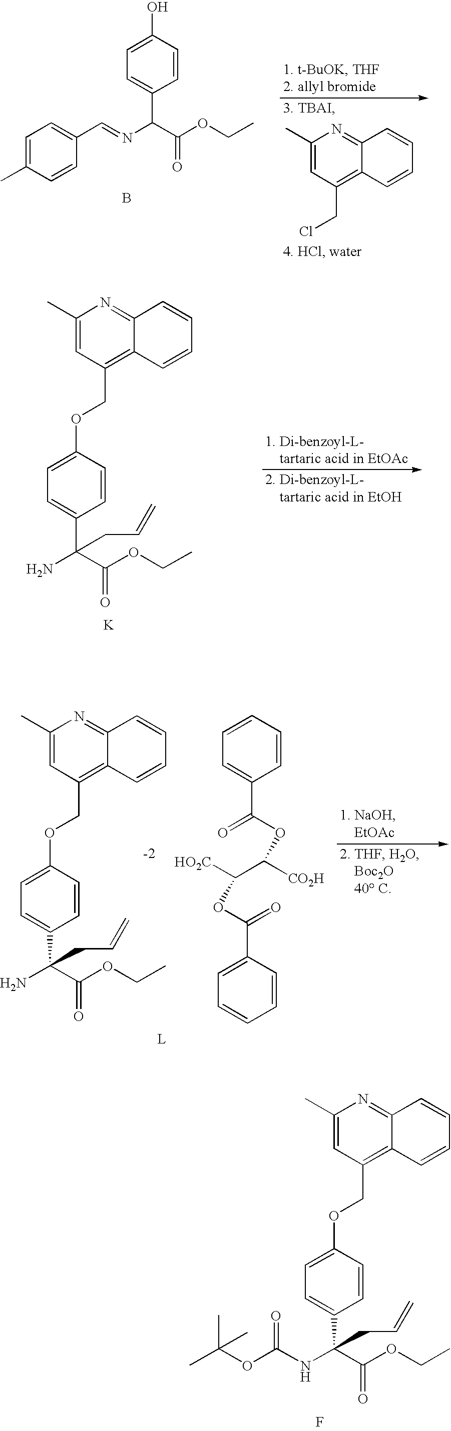

Preparation of Compound L from Compound B

A 500 mL flask was charged with Compound B (15.0 g, 50 mmol) and tetrahydrofuran (150 mL, THF) and cooled to −10° C. Allyl bromide (6.4 g, 53 mmol, 1.06 eq) was charged, followed by potassium tert-butoxide (12.2 g, 109 mmol, 2.2 eq) at −10 to 0° C. over 30 minutes. After holding for 30 minutes at approximately −5° C., the reaction mass was sampled for conversion and determined complete by HPLC. 4-Chloromethyl-2-methylquinoline (9.6 g, 50 mmol, 1 eq) and tetrabutylammonium iodide (0.9 g, 2.5 mmol, 0.5 eq) were charged and the reaction was heated to 40° C. After holding for 60 minutes at ˜40° C., the reaction mass was sampled for conversion and determined complete by HPLC. The reaction was quenched by adding 1 M aqueous hydrochloric acid (150 ml, 150 mmol, 3 eq), pH=1 after quench. Heptanes (100 mL) were added and the layers were separated, retaining the product rich aqueous phase. Ethyl acetate (150 mL) was added and the pH of the aqueous phase was adjusted to 8 by adding a saturated aqueous sodium bicarbonate solution. The layers were separated, retaining the product rich organic phase.

Ethyl acetate was added to adjust the final volume to 300 mL. The solution was heated to 70° C. and dibenzoyl-L-tartaric acid (14.5 g, 40 mmol, 0.8 eq) in 2-propyl alcohol (50 mL) was added. The reaction was seeded with the undesired salt and cooled to 20° C. over approximately 3 h. The undesired salt (15.1 g, 80% ee) was removed leaving the product rich filtrate (−40% ee). After removing the ethyl acetate, the filtrate was dissolved in 3:1 IPA:MeOH (300 mL) and heated to 70° C. Dibenzoyl-L-tartaric acid (15.0 g, 41.8 mmol) was then added and the solution was held at 70° C. until clear. Seed crystals were added (99% ee) and the solution was cooled to 50° C. After 1 h, the slurry was cooled to ˜25° C. and filtered. The cake was washed with ethyl alcohol (2×40 mL) and dried in a vacuum oven at 50° C. to a constant weight (18.5 g, 80% ee). The product was then dissolved in MeOH (80 mL) and EtOH (80 mL) at 70° C. ethyl alcohol (160 mL) was then added over 30 minutes. The solution was cooled to ˜25° C. and filtered.

The product was washed with EtOH (2×30 mL) and dried to a constant weight to yield 15.6 g Compound L, 28% yield from Compound B, 91% ee dibenzoyl-L-tartaric acid salt as a white solid. ¹H NMR (400 MHz, DMSO d₆) δ 8.12 (1H, d, J=8.1 Hz), 8.02 (8H, d, J=7.6 Hz), 7.99 (1H, m), 7.76 (1H, t, J=7.1 Hz), 7.71-7.68 (4H, m), 7.61-7.51 (10H, m), 7.47 (2H, d, J=8.6 Hz), 7.20 (2H, d, J=8.6 Hz), 5.81 (4H, s), 5.80-5.70 (2H, m), 5.64 (2H, s), 5.30-5.12 (2H, m), 4.29-4.09 (2H, m), 3.54-3.36 (2H, m), 2.67 (3H, s), 1.17 (3H, t, J=7.1 Hz). ¹³C NMR (100 MHz, DMSO d₆) δ 170.5, 167.9, 165.2, 158.9, 158.6, 147.6, 142.4, 134.1, 130.9, 129.8, 129.7, 129.4, 129.2, 129.1, 128.1, 126.3, 124.3, 124.1, 121.8, 120.5, 115.3, 72.4, 66.7, 64.0, 62.7, 40.6, 25.3, 14.2.

Preparation of Compound F from Compound L

A 1 L flask was charged sequentially with Compound L (31 g, 28 mmol), EtOAc (200 mL), and water (200 mL). 5M NaOH was added to adjust to pH 9-10. The aqueous layer was discarded and the organic layer was washed with water (200 mL). The organic layer was then concentrated and dissolved in THF (50 mL). Water (50 mL), di-tert-butyl dicarbonate (12 g, 55 mmol) and triethylamine (2.8 g, 28 mmol) were added and the reaction mixture heated to 40° C. After 48 h, the reaction was deemed complete by HPLC (>95% conversion). Ethyl acetate (100 mL) was charged and the aqueous layer was removed. The organic layer was then washed with water (2×50 mL) and the solvent was removed to afford 14.2 g compound L as an oil.

### Utility

The compounds of formula I are expected to possess matrix metalloprotease and/or TNF-α inhibitory activity. The MMP inhibitory activity of the compounds of the present invention is demonstrated using assays of MMP activity, for example, using the assay described below for assaying inhibitors of MMP activity. The compounds of the present invention are expected to be bioavailable in vivo as demonstrated, for example, using the ex vivo assay described below. The compounds of formula I are expected to have the ability to suppress/inhibit cartilage degradation in vivo, for example, as demonstrated using the animal model of acute cartilage degradation described below.

The compounds provided by this invention should also be useful as standards and reagents in determining the ability of a potential pharmaceutical to inhibit MPs. These would be provided in commercial kits comprising a compound of this invention.

Metalloproteinases have also been implicated in the degradation of basement membranes to allow infiltration of cancer cells into the circulation and subsequent penetration into other tissues leading to tumor metastasis (Stetler-Stevenson, Cancer and Metastasis Reviews, 9, 289-303, 1990). The compounds of the present invention should be useful for the prevention and treatment of invasive tumors by inhibition of this aspect of metastasis.

The compounds of the present invention should also have utility for the prevention and treatment of osteopenia associated with matrix metalloprotease-mediated breakdown of cartilage and bone that occurs in osteoporosis patients.

Compounds that inhibit the production or action of TACE and/or MMP's are potentially useful for the treatment or prophylaxis of various inflammatory, infectious, immunological or malignant diseases or conditions. Thus, the present invention relates to a method of treating various inflammatory, infectious, immunological or malignant diseases. These include acute infection, acute phase response, age related macular degeneration, alcoholic liver disease, allergy, allergic asthma, anorexia, aneurism, aortic aneurism, asthma, atherosclerosis, atopic dermatitis, autoimmune disease, autoimmune hepatitis, Bechet's disease, cachexia (including cachexia resulting from cancer or HIV), calcium pyrophosphate dihydrate deposition disease, cardiovascular effects, chronic fatigue syndrome, chronic obstruction pulmonary disease, coagulation, congestive heart failure, corneal ulceration, Crohn's disease, enteropathic arthropathy (including inflammatory bowl disease), Felty's syndrome, fever, fibromyalgia syndrome, fibrotic disease, gingivitis, glucocorticoid withdrawal syndrome, gout, graft versus host disease, hemorrhage, HIV infection, hyperoxic alveolar injury, infectious arthritis, inflammation, intermittent hydrarthrosis, Lyme disease, meningitis, multiple sclerosis, myasthenia gravis, mycobacterial infection, neovascular glaucoma, osteoarthritis, pelvic inflammatory disease, periodontitis, polymyositis/dermatomyositis, post-ischaemic reperfusion injury, post-radiation asthenia, psoriasis, psoriatic arthritis, pulmonary emphysema, pydoderma gangrenosum, relapsing polychondritis, Reiter's syndrome, rheumatic fever, rheumatoid arthritis (including juvenile rheumatoid arthritis and adult rheumatoid arthritis), sarcoidosis, scleroderma, sepsis syndrome, Still's disease, shock, Sjogren's syndrome, skin inflammatory diseases, solid tumor growth and tumor invasion by secondary metastases, spondylitis, stroke, systemic lupus erythematosus, ulcerative colitis, uveitis, vasculitis, and Wegener's granulomatosis.

Some compounds of the present invention have been shown to inhibit TNF production in lipopolysacharride stimulated mice, for example, using the assay for TNF induction in mice and in human whole blood as described below.

The compounds of the present invention can be administered alone or in combination with one or more additional anti-inflammatory agents. These agents include, but are not limited to, selective COX-2 inhibitors, interleukin-1 antagonists, dihydroorotate synthase inhibitors, p38 MAP kinase inhibitors, TNF-α inhibitors, and TNF-α sequestration agents.

By “administered in combination” or “combination therapy” it is meant that a compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

The term selective COX-2 inhibitors, as used herein, denote agents that selectively inhibit COX-2 function. Such agents include, but are not limited to, celecoxib (Celebrex®), rofecoxib (Vioxx®), meloxicam (Movicox®), etoricoxib, and valdecoxib.

TNF-α sequestration agents that may be used in combination with the compounds of this invention, are TNF-α binding proteins or anti-TNF-α antibodies. These agents include, but are not limited to, etanercept (Enbrel®), infliximab (Remicade®), adalimumab (D2E7), CDP-571 (Humicade®), and CDP-870.

Other anti-inflammatory agents that may be used in combination with the compounds of this invention, include, but are not limited to, methotrexate, interleukin-1 antagonists (e.g., anakinra (Kineret®)), dihydroorotate synthase inhibitors (e.g., leflunomide (Arava®)), and p38 MAP kinase inhibitors.

Administration of the compounds of the present invention in combination with such additional therapeutic agent, may afford an efficacy advantage over the compounds and agents alone, and may do so while permitting the use of lower doses of each. A lower dosage minimizes the potential of side effects, thereby providing an increased margin of safety.

A compound is considered to be active if it has an IC₅₀ or Kᵢ value of less than about 10 μM for the inhibition of a desired MP. Preferred compounds of the present invention have Kᵢ's or IC₅₀'s of ≦1 μM. More preferred compounds of the present invention have Kᵢ's or IC₅₀'s of ≦0.1 μM. Even more preferred compounds of the present invention have Kᵢ's or IC₅₀'s of ≦0.01 μM. Still more preferred compounds of the present invention have Kᵢ's or IC₅₀'s of ≦0.001 μM.

TNF PBMC Assay

Human peripheral blood mononuclear cells (PBMC) were obtained from normal donor blood by leukophoresis and isolated by Ficoll-Paque density separation. PBMCs were suspended in 0.5 mL RPMI 1640 with no serum at 2×10⁶ cells/mL in 96 well polystyrene plates. Cells were preincubated 10 minutes with compound, and then stimulated with 1 μg/mL LPS (Lipopolysaccharide, Salmonella typhimurium) to induce TNF production. After an incubation of 5 h at 37° C. in 95% air, 5% CO₂ environment, culture supernatants were removed and tested by standard sandwich ELISA for TNF production.

TNF Human Whole Blood Assay

Blood is drawn from normal donors into tubes containing 143 USP units of heparin/10 mL. 225 μL of blood is plated directly into sterile polypropylene tubes. Compounds are diluted in DMSO/serum free media and added to the blood samples so the final concentration of compounds are 50, 10, 5, 1, 0.5, 0.1, and 0.01 μM. The final concentration of DMSO does not exceed 0.5%. Compounds are preincubated for 15 minutes before the addition of 100 mg/mL LPS. Plates are incubated for 5 h in an atmosphere of 5% CO₂ in air. At the end of 5 h, 750 uL of serum free media is added to each tube and the samples are spun at 1200 RPM for 10 minutes. The supernatant is collected off the top and assayed for TNF-alpha production by a standard sandwich ELISA. The ability of compounds to inhibit TNF-alpha production by 50% compared to DMSO treated cultures is given by the IC₅₀ value.

TNF Induction in Mice

Test compounds are administered to mice either I.P. or P.O. at time zero. Immediately following compound administration, mice receive an I.P. injection of 20 mg of D-galactosamine plus 10 μg of lipopolysaccharide. One hour later, animals are anesthetized and bled by cardiac puncture. An ELISA specific for mouse TNF evaluates blood plasma for TNF levels. Administration of representative compounds of the present invention to mice results in a dose-dependent suppression of plasma TNF levels at one hour in the above assay.

MMP ASSAYS

The enzymatic activities of recombinant MMP-1, 2, 3, 7, 8, 9, 10, 12, 13, 14, 15, and 16 were measured at 25° C. with a fluorometric assay (Copeland, R. A. et al. Bioorganic Med. Chem. Lett. 1995, 5, 1947-1952). Final enzyme concentrations in the assay were between 0.05 and 10 nM depending on the enzyme and the potency of the inhibitor tested. The permisive peptide substrate, MCA-Pro-Leu-Gly-Leu-DPA-Ala-Arg-NH₂, was present at a final concentration of 10 μM in all assays. Initial velocities, in the presence or absence of inhibitor, were measured as slopes of the linear portion of the product progress curves. IC50 values were determined by plotting the inhibitor concentration dependence of the fractional velocity for each enzyme, and fitting the data by non-linear least squares methods to the standard isotherm equation (Copeland, R. A. Enzymes: A practical Introduction to Structure, Mechanism and Data Analysis, Wiley-VHC, New York, 1996, pp 187-223). All of the compounds studied here were assumed to act as competitive inhibitors of the enzyme, binding to the active site Zn atom as previously demonstrated by crystallographic studies of MMP-3 complexed with related hydroxamic acids (Rockwell, A. et al. J. Am. Chem. Soc. 1996, 118, 10337-10338). Based on the assumption of competitive inhibition, the IC50 values were converted to Kᵢ values as previously described.

Compounds tested in the above assay are considered to be active if they exhibit a Kᵢ of ≦10 μM. Preferred compounds of the present invention have Kᵢ's of ≦1 μM. More preferred compounds of the present invention have Kᵢ's of ≦0.1 μM. Even more preferred compounds of the present invention have Kᵢ's of ≦0.01 μM. Still more preferred compounds of the present invention have Kᵢ's of ≦0.001 μM.

### Dosage and Formulation

The compounds of the present invention can be administered orally using any pharmaceutically acceptable dosage form known in the art for such administration. The active ingredient can be supplied in solid dosage forms such as dry powders, granules, tablets or capsules, or in liquid dosage forms, such as syrups or aqueous suspensions. The active ingredient can be administered alone, but is generally administered with a pharmaceutical carrier. A valuable treatise with respect to pharmaceutical dosage forms is Remington's Pharmaceutical Sciences, Mack Publishing.

The compounds of the present invention can be administered in such oral dosage forms as tablets, capsules (each of which includes sustained release or timed release formulations), pills, powders, granules, elixirs, tinctures, suspensions, syrups, and emulsions. Likewise, they may also be administered in intravenous (bolus or infusion), intraperitoneal, subcutaneous, or intramuscular form, all using dosage forms well known to those of ordinary skill in the pharmaceutical arts. An effective but non-toxic amount of the compound desired can be employed as an antiinflammatory and antiarthritic agent.

The compounds of this invention can be administered by any means that produces contact of the active agent with the agent's site of action in the body of a mammal. They can be administered by any conventional means available for use in conjunction with pharmaceuticals, either as individual therapeutic agents or in a combination of therapeutic agents. They can be administered alone, but generally administered with a pharmaceutical carrier selected on the basis of the chosen route of administration and standard pharmaceutical practice.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. An ordinarily skilled physician or veterinarian can readily determine and prescribe the effective amount of the drug required to prevent, counter, or arrest the progress of the condition.

By way of general guidance, the daily oral dosage of each active ingredient, when used for the indicated effects, will range between about 0.001, 0.01, 0.1, 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, to 1000 mg/kg of body weight, preferably between about 0.01 to 100 mg/kg of body weight per day, and most preferably between about 1.0 to 20 mg/kg/day. For a normal male adult human of approximately 70 kg of body weight, this translates into a dosage of 70 to 1400 mg/day. Intravenously, the most preferred doses will range from about 1 to about 10 mg/kg/minute during a constant rate infusion. Advantageously, compounds of the present invention may be administered in a single daily dose, or the total daily dosage may be administered in divided doses of two, three, or four times daily.

The compounds for the present invention can be administered in intranasal form via topical use of suitable intranasal vehicles, or via transdermal routes, using those forms of transdermal skin patches wall known to those of ordinary skill in that art. To be administered in the form of a transdermal delivery system, the dosage administration will, of course, be continuous rather than intermittent throughout the dosage regimen.

In the methods of the present invention, the compounds herein described in detail can form the active ingredient, and are typically administered in admixture with suitable pharmaceutical diluents, excipients, or carriers (collectively referred to herein as carrier materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 1 milligram to about 100 milligrams of active ingredient per dosage unit. In these pharmaceutical compositions the active ingredient will ordinarily be present in an amount of about 0.5-95% by weight based on the total weight of the composition.

The active ingredient can be administered orally in solid dosage forms, such as capsules, tablets, and powders, or in liquid dosage forms, such as elixirs, syrups, and suspensions. It can also be administered parenterally, in sterile liquid dosage forms.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

The compounds of the present invention may be administered in combination with a second therapeutic agent, especially non-steroidal anti-inflammatory drugs (NSAID's). The compound of Formula I and the second therapeutic agent can be administered separately or as a physical combination in a single dosage unit, in any dosage form and by various routes of administration, as described above.

The compound of Formula I may be formulated together with the second therapeutic agent in a single dosage unit (that is, combined together in one capsule, tablet, powder, or liquid, etc.). When the compound of Formula I and the second therapeutic agent are not formulated together in a single dosage unit, the compound of Formula I and the second therapeutic agent may be administered essentially at the same time, or in any order; for example the compound of Formula I may be administered first, followed by administration of the second agent. When not administered at the same time, preferably the administration of the compound of Formula I and the second therapeutic agent occurs less than about one hour apart, more preferably less than about 5 to 30 minutes apart.

Preferably the route of administration of the compound of Formula I is oral. Although it is preferable that the compound of Formula I and the second therapeutic agent are both administered by the same route (that is, for example, both orally), if desired, they may each be administered by different routes and in different dosage forms (that is, for example, one component of the combination product may be administered orally, and another component may be administered intravenously).

The dosage of the compound of Formula I when administered alone or in combination with a second therapeutic agent may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired, as described above.

Particularly when provided as a single dosage unit, the potential exists for a chemical interaction between the combined active ingredients. For this reason, when the compound of Formula I and a second therapeutic agent are combined in a single dosage unit they are formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a sustained-release material that affects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low-viscosity grade of hydroxypropyl methylcellulose (HPMC) or other appropriate materials as known in the art, in order to further separate the active components. The polymer coating serves to form an additional barrier to interaction with the other component.

These as well as other ways of minimizing contact between the components of combination products of the present invention, whether administered in a single dosage form or administered in separate forms but at the same time by the same manner, will be readily apparent to those skilled in the art, once armed with the present disclosure.

The present invention also includes pharmaceutical kits useful, for example, in the treatment or prevention of osteoarthritis or rheumatoid arthritis, which comprise one or more containers containing a pharmaceutical composition comprising a therapeutically effective amount of a compound of Formula I. Such kits may further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, may also be included in the kit.

In the present disclosure it should be understood that the specified materials and conditions are important in practicing the invention but that unspecified materials and conditions are not excluded so long as they do not prevent the benefits of the invention from being realized.

Although this invention has been described with respect to specific embodiments, the details of these embodiments are not to be construed as limitations. Various equivalents, changes and modifications may be made without departing from the spirit and scope of this invention, and it is understood that such equivalent embodiments are part of this invention.

What is claimed is:

1. A process, comprising:
(a) contacting a compound of formula I with a base and a halo-allyl group to form a compound of formula II: 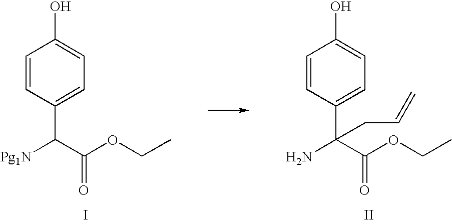
wherein: Pg₁ is an amino protecting group or represents H and an amino protecting group; and,
the ratio of carbon to oxygen alkylation is from about 50-1000.

2. A process according to claim 1, further comprising: (b) resolving a compound of formula II to form a compound of formula III: 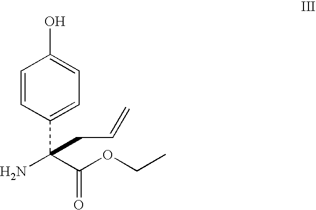
wherein: the base for (a) is lithium t-butoxide;
the allyl-halo reagent is allyl bromide; and,
Pg₁ is 4-methylbenzaldehyde.

3. A process according to claim 2, wherein (b) is performed by contacting a compound of formula II with pig liver esterase and the enantiomeric excess of the R-isomer of formula III is at least 90%.

4. A process according to claim 2, further comprising:
(c) protecting the amino group of the compound of formula III to form a compound of formula IV: 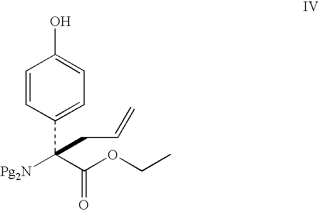
wherein Pg₂ is an amino protecting group or represents H and an amino protecting group.

5. A process according to claim 4, wherein (c) is performed by contacting a compound of formula III with an amino protecting reagent in the presence of a base;
wherein the amino protecting group reagent is selected form di-tert-butyl dicarbonate and 2-(tert-butoxycarbonyloxyimino)-2-phenylacetonitrile; and,
the base is selected from lithium carbonate, sodium carbonate, and potassium carbonate.

6. A process according to claim 5, wherein:
Pg₂ is tert-butoxycarbonyl;
the amino protecting group reagent is di-tert-butyl dicarbonate; and,
the base is lithium carbonate.

7. A process according to claim 4, further comprising:
(d) contacting a compound of formula IV with an alkylating agent to form a compound of formula V:

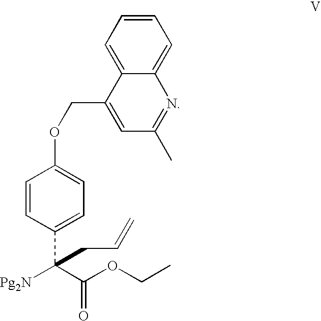

8. A process according to claim 7, wherein the alkylating agent is a 4-halomethyl-2-methylquinoline and the contacting is done in the presence of a base.

9. A process according to claim 8, wherein the alkylating agent is 4-chloromethyl-2-methylquinoline and the base is potassium tert-butoxide.

10. A process according to claim 7, further comprising:
(e) cleaving the olefin of formula V to form a compound of formula VI; and, 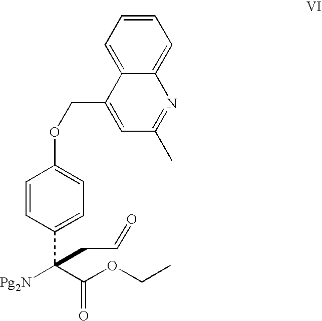
(f) contacting the compound of formula VI with an ester of an amino acid to form a compound of formula VII; 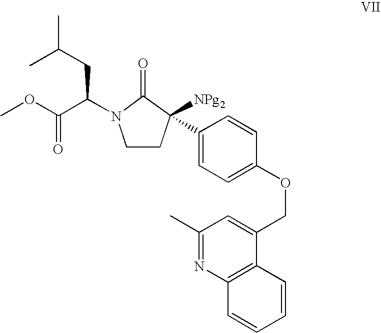
wherein (f) is done in the presence of a reducing agent.

11. A process according to claim 10, further comprising:
(g) deprotecting the compound of formula VII to form a compound of formula VIII.

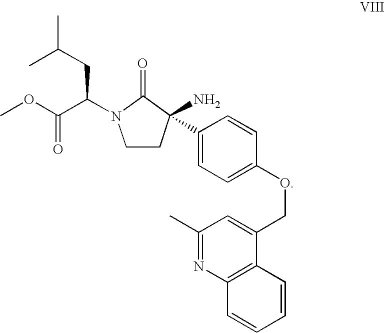

12. A compound of formula III: 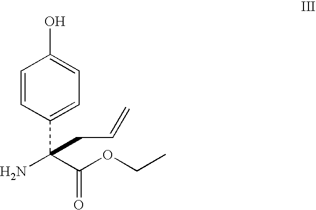
or a salt form thereof.

13. A compound according to claim 12, wherein the compound of formula III is a methanesulfonic acid salt.

14. A compound of formula IV: 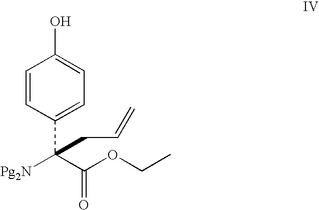
wherein Pg₂ is an amino protecting group or represents H and an amino protecting group.

15. A compound according to claim 14, wherein Pg₂ is tert-butoxycarbonyl.

16. A compound of formula VIII or a salt form thereof:

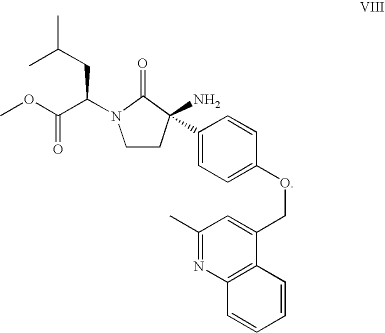

17. A compound according to claim 16, wherein the compound of formula VIII is a bis-methanesulfonic acid salt.

18. A crystalline, free-base form of Compound J ((2R)-2-((3R)-3-amino-3-{-[(2-methyl-4-quinolinyl)methoxy]phenyl}-2-oxopyrrolidinyl)-N-hydroxy-4-methylpentanamide):

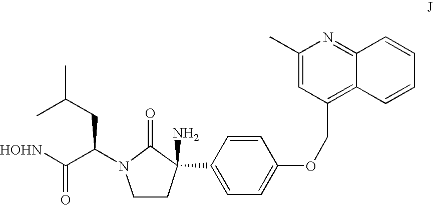

19. The compound according to claim 18, wherein the compound is characterized by an x-ray powder diffraction pattern substantially in accordance with that shown in FIG. 1.

20. The compound according to claim 18, wherein the compound is characterized by a differential scanning calorimetry thermogram substantially in accordance with that shown in FIG. 2.

21. A compound according to claim 18, wherein the compound is characterized by a differential scanning calorimetry thermogram having a melt onset at about 194.4±0.5° C. with melting peak at about 195.9±0.5° C. followed by decomposition, wherein the DSC is operated at a rate of about 10° C./minute.

22. A compound according to claim 18, wherein the compound is characterized by an x-ray powder diffraction pattern with its most intense reflections comprising the following 20 values 6.7±0.2; 8.4±0.2; 9.2±0.2; 13.5±0.2; 14.2±0.2; 16.7±0.2; 17.4±0.2; 19.6±0.2; 19.9±0.2; 20.1±0.2; 20.9±0.2; and, 22.6±0.2 and a differential scanning calorimetry thermogram substantially in accordance with that shown in FIG. 2.

23. A pharmaceutical composition, comprising: a pharmaceutically acceptable carrier and a therapeutically effective amount of a compound according to claim 18 or a pharmaceutically acceptable salt form thereof.

24. A method for treating an inflammatory disorder, comprising: administering to a patient in need thereof a therapeutically effective amount of a compound according to claim 18 or a pharmaceutically acceptable salt form thereof.

25. A method of treating a condition or disease mediated by MMPs, TACE, or a combination thereof in a mammal, comprising: administering to the mammal in need of such treatment a therapeutically effective amount of a compound according to claim 18 or a pharmaceutically acceptable salt form thereof.

26. A method comprising: administering a compound according to claim 18 or a pharmaceutically acceptable salt form thereof, in an amount effective to treat a condition or disease mediated by MMPs, TACE, or a combination thereof.

27. A method of treating a disease or condition according to claim 25, wherein the disease or condition is selected from to as acute infection, acute phase response, age related macular degeneration, alcoholic liver disease, allergy, allergic asthma, anorexia, aneurism, aortic aneurism, asthma, atherosclerosis, atopic dermatitis, autoimmune disease, autoimmune hepatitis, Bechet's disease, cachexia, calcium pyrophosphate dihydrate deposition disease, cardiovascular effects, chronic fatigue syndrome, chronic obstruction pulmonary disease, coagulation, congestive heart failure, corneal ulceration, Crohn's disease, enteropathic arthropathy, Felty's syndrome, fever, fibromyalgia syndrome, fibrotic disease, gingivitis, glucocorticoid withdrawal syndrome, gout, graft versus host disease, hemorrhage, HIV infection, hyperoxic alveolar injury, infectious arthritis, inflammation, intermittent hydrarthrosis, Lyme disease, meningitis, multiple sclerosis, myasthenia gravis, mycobacterial infection, neovascular glaucoma, osteoarthritis, pelvic inflammatory disease, periodontitis, polymyositis/dermatomyositis, post-ischaemic reperfusion injury, post-radiation asthenia, psoriasis, psoriatic arthritis, pulmonary emphysema, pydoderma gangrenosum, relapsing polychondritis, Reiter's syndrome, rheumatic fever, rheumatoid arthritis, sarcoidosis, scleroderma, sepsis syndrome, Still's disease, shock, Sjogren's syndrome, skin inflammatory diseases, solid tumor growth and tumor invasion by secondary metastases, spondylitis, stroke, systemic lupus erythematosus, ulcerative colitis, uveitis, vasculitis, and Wegener's granulomatosis.

28. A method for treating an inflammatory disorder, comprising: administering, in combination, to a host in need thereof, a therapeutically effective amount of
(a) a compound of claim 18 or a pharmaceutically acceptable salt form thereof; and,
(b) one or more additional anti-inflammatory agents selected from selective COX-2 inhibitors, interleukin-1 antagonists, dihydroorotate synthase inhibitors, p38 MAP kinase inhibitors, TNF-α inhibitors, TNF-α sequestration agents, and methotrexate.
